SEQUENCE LISTING (34 sequences)

SEQ ID NO: 1 - RNA, 23 nt, homo sapiens
ucuuugguuaucuagcuguauga

SEQ ID NO: 2 - RNA, 22 nt, homo sapiens
uagcagcacguaaauauuggcg

SEQ ID NO: 3 - RNA, 22 nt, homo sapiens
aagcugccaguugaagaacugu

SEQ ID NO: 4 - RNA, 23 nt, homo sapiens
gugccuacugagcugauaucagu

SEQ ID NO: 5 - RNA, 22 nt, homo sapiens
cauugcacuugucucggucuga

SEQ ID NO: 6 - RNA, 22 nt, homo sapiens
aaggagcucacagucuauugag

SEQ ID NO: 7 - RNA, 22 nt, homo sapiens
uguaaacauccucgacuggaag

SEQ ID NO: 8 - RNA, 22 nt, homo sapiens
aaagugcuguucgugcagguag

SEQ ID NO: 9 - RNA, 22 nt, homo sapiens
aacccguagauccgaacuugug

SEQ ID NO: 10 - RNA, 23 nt, homo sapiens
agcagcauuguacagggcuauga

SEQ ID NO: 11 - RNA, 23 nt, homo sapiens
agcagcauuguacagggcuauca

SEQ ID NO: 12 - RNA, 23 nt, homo sapiens
uggagugugacaaugguguuugu

SEQ ID NO: 13 - RNA, 22 nt, homo sapiens
uuaaggcacgcggugaaugcca

SEQ ID NO: 14 - RNA, 21 nt, homo sapiens
cauuauuacuuuugguacgcg

SEQ ID NO: 15 - RNA, 22 nt, homo sapiens
uaacagucuacagccauggucg

SEQ ID NO: 16 - RNA, 23 nt, homo sapiens
acuccauuuguuuugaugaugga

SEQ ID NO: 17 - RNA, 21 nt, homo sapiens
agugguuuuacccuaugguag

SEQ ID NO: 18 - RNA, 20 nt, homo sapiens
cauaaaguagaaagcacuac

SEQ ID NO: 19 - RNA, 20 nt, homo sapiens
cauaaaguagaaagcacuac

SEQ ID NO: 20 - RNA, 22 nt, homo sapiens
ugagaugaagcacuguagcuca

SEQ ID NO: 21 - RNA, 24 nt, homo sapiens
guccaguuuucccaggaaucccuu

SEQ ID NO: 22 - RNA, 22 nt, homo sapiens
ucuggcuccgugucuucacucc

SEQ ID NO: 23 - RNA, 22 nt, homo sapiens
ucagugcaugacagaacuuggg

SEQ ID NO: 24 - RNA, 22 nt, homo sapiens
uagguuauccguguugccuucg

SEQ ID NO: 25 - RNA, 21 nt, homo sapiens
ucgugucuuguguugcagccg

SEQ ID NO: 26 - RNA, 22 nt, homo sapiens
caacggaaucccaaaagcagcu

SEQ ID NO: 27 - RNA, 21 nt, homo sapiens
uagcagcacagaaauauuggc

SEQ ID NO: 28 - RNA, 22 nt, homo sapiens
uccuucauuccaccggagucug

SEQ ID NO: 29 - RNA, 22 nt, homo sapiens
uggaauguaaggaagugugugg

SEQ ID NO: 30 - RNA, 22 nt, homo sapiens
cugugcgugugacagcggcuga

SEQ ID NO: 31 - RNA, 20 nt, homo sapiens
cauaaaguagaaagcacuac

SEQ ID NO: 32 - RNA, 21 nt, homo sapiens
uaaucucagcuggcaacugug

SEQ ID NO: 33 - RNA, 21 nt, homo sapiens
ugauuguccaaacgcaauucu

SEQ ID NO: 34 - RNA, 23 nt, homo sapiens
agcuacauugucugcuggguuuc

(19) United States
(12) Patent Application Publication
(10) Pub. No.: US 2006/0292616 A1
(43) Pub. Date: Dec. 28, 2006

(54) Single molecule miRNA-based disease diagnostic methods

(75) Inventors: Lori A. Neely, Reading, MA (US); Sonal Patel, Nashua, NH (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: U.S. Genomics, Inc., Woburn, MA (US)

(21) Appl. No.: 11/474,067

(22) Filed: Jun. 23, 2006

Related U.S. Application Data
(60) Provisional application No. 60/693,333, filed on Jun. 23, 2005.

(51) Int. Cl. C12Q 1/68

(52) U.S. Cl. 435/6

(57) ABSTRACT

The invention relates to compositions and methods for detecting and quantifying small RNA species such as miRNA, preferably associated with disease detection and diagnosis.

### RELATED APPLICATIONS

This application claims priority to U.S. Provisional Application having Ser. No. 60/693,333, and entitled “SINGLE MOLECULE miRNA-BASED DISEASE DIAGNOSTIC METHODS”, filed on Jun. 23, 2005, the entire contents of which are incorporated by reference herein.

### FIELD OF THE INVENTION

The invention provides methods and compositions for analysis of microRNA, including detection and quantitation.

### BACKGROUND OF THE INVENTION

Short non-coding RNA molecules are potent regulators of gene expression. First discovered in C. elegans (Lee 1993) these highly conserved endogenously expressed ribo-regulators are called microRNAs (miRNAs). miRNAs are short naturally occurring RNAs generally ranging in length from about 7 to about 27 nucleotides.

Only a few hundred miRNAs have been identified. This number is far lower than the expected number of coding sequences in the human genome. However, it is not expected that each coding sequence has its own unique miRNA. This is because miRNAs generally hybridize to RNAs with one or more mismatches. The ability of the miRNA to bind to RNA targets in spite of these apparent mismatches provides the variability necessary to potentially modulate a number of transcripts with a single miRNA.

miRNA therefore can act as regulators of cellular development, differentiation, proliferation and apoptosis. miRNAs can modulate gene expression by either impeding mRNA translation, degrading complementary mRNAs, or targeting genomic DNA for methylation. For example, miRNAs can modulate translation of mRNA transcripts by binding to and thereby making such transcripts susceptible to nucleases that recognize and cleave double stranded RNAs. miRNAs have also been implicated as developmental regulators in mammals in two recent mouse studies characterizing specific miRNAs involved in stem cell differentiation (Houbaviy H B 2003; Chen C Z 2004). Numerous studies have demonstrated miRNAs are critical for cell fate commitment and cell proliferation (Brennecke J 2003) (Zhao Y 2005). Other studies have analyzed the role of miRNAs in cancer (Michael M Z 2003; Calin 2004; He 2005; Johnson S M 2005). miRNAs may play a role in diabetes (Poy M N 2004) and neurodegeneration associated with Fragile X syndrome, spinal muscular atrophy, and early on-set Parkinson's disease (Caudy 2002; Hutvagner 2002; Mouelatos 2002; Dostie 2003). Several miRNAs are virally encoded and expressed in infected cells (e.g., EBV, HPV and HCV).

Analysis of the role of miRNA in these processes, as well as other applications, would be aided by the ability to more accurately and specifically detect and measure miRNA. However, the short nature of the miRNAs makes them difficult to quantify using conventional prior art methods. For example, although Northern blotting has been the “gold standard” for miRNA quantification, this technique is limited in its sensitivity, throughput, and reproducibility. In addition, Northern blotting requires 10-30 micrograms of tissue total RNA and a typical experiment takes 24 to 48 hours to perform with long incubations required for probe hybridization and blot exposure.

There exists a need for methods and systems for detecting and quantitating miRNA, preferably without the need for nucleic acid amplification. Such methods are preferably robust, specific and sufficiently sensitive to abolish the need for amplification.

### SUMMARY OF THE INVENTION

The invention relates in part to direct quantification of small non-coding RNAs (e.g., miRNAs). Such quantification may be performed at the single molecule level. The detection and quantification of these RNAs is used in the identification and characterization of human disease.

In one aspect, the invention provides a method for diagnosing a condition comprising determining a level of a miRNA in a test tissue sample, and comparing the level of the miRNA in the test tissue sample to a level of the miRNA in a control tissue sample.

A difference in the level of the miRNA in the test and the control tissue samples is indicative of the condition. In one embodiment, the difference in the level of the miRNA in the test and the control tissue samples is a greater level of miRNA in the test tissue sample. In another embodiment, the difference in the level of the miRNA in the test and the control tissue samples is a greater level of miRNA in the control tissue sample.

The level of the miRNA is determined by coincidence binding of one or more probes to the target miRNA. If more than one probe is used, preferably the probes are differentially and detectably labeled. The coincidence binding is performed at a single molecule level. In an important embodiment, coincidence binding comprises coincident detection of for example two signals from a first miRNA-specific probe labeled with a first detectable label and a second miRNA-specific probe labeled with a second detectable label distinguishable from the first detectable label. Such analysis may further comprise subtracting a random coincidence estimate from a raw coincidence count. In yet another embodiment, coincidence binding comprises use of a quencher probe.

In one embodiment, the test tissue sample is a breast tissue sample, a cervical tissue sample, an ovarian tissue sample, or a prostate tissue sample.

In one embodiment, the condition is cancer such as but not limited to breast cancer, cervical cancer, colon cancer, ovarian cancer, or prostate cancer. In another embodiment, the condition is cirrhosis.

In one embodiment, the miRNA is mir-143 or mir-145. Preferably, the miRNA is a human miRNA and the samples are human samples.

In certain embodiment, the miRNA is present at a concentration of 1-1000 femtomolar, 1-100 femtomolar, or 1-10 femtomolar.

In another aspect, the invention provides a method for detecting microRNA in a sample comprising contacting a sample with a first and a second nucleic acid probe under conditions and for a time sufficient to allow hybridization to a microRNA, wherein the first and second nucleic acid probes are conjugated to a first and second detectable label, respectively, that are distinct from each other, and detecting coincident binding of the first and second nucleic acid probes to a single microRNA as coincident signals from the first and second detectable labels. Hybridization of the first and second nucleic acid probes to a microRNA results in a double stranded duplex (or hybrid) having at least a one or two base overhang at the 3′ and 5′ end of the microRNA, and coincident signals are indicative of a microRNA.

In one embodiment, the first and second nucleic acid probes have a sum total length that is at least 2, at least 3, at least 4, or at least 5 bases longer than the microRNA. In one embodiment, the first and second nucleic acid probes each is a DNA, PNA, LNA or a combination thereof.

In one embodiment, the first nucleic acid probe is conjugated to a first fluorophore and the second nucleic acid probe is conjugated to a second fluorophore and the first and second fluorophores are a FRET pair.

In one embodiment, the one or two base overhang at the 3′ and 5′ end each comprises a cytosine or a guanosine. In one embodiment, the one or two base overhang at the 3′ and 5′ end each comprises an adenine or a thymidine. In one embodiment, the one or two base overhang at the 3′ and 5′ end each comprises an iso-guanosine or an iso-cytosine.

In one embodiment, the first and second nucleic acid probes is each at least 12 or at least 13 bases long. In one embodiment, the first and second nucleic acid probes have a sum total length that is greater than the length of the microRNA.

In one embodiment, the method further comprises isolating the double stranded duplex from the sample.

In one embodiment, the double stranded duplex is isolated from the sample by size separation. In one embodiment, the sample comprises a plurality of RNA molecules.

In one embodiment, the method further comprises column purification prior to detecting coincident binding. In one embodiment, the method further comprises addition of a quencher labeled nucleic acid probe to the sample prior to detecting coincident binding. In one embodiment, the method further comprises addition of a single stranded nuclease to the sample prior to detecting coincident binding.

In one embodiment, the method further comprises ligating the first nucleic acid probe to the second nucleic acid probe prior to detecting coincident binding.

In another aspect, the invention provides a method for detecting microRNA in sample comprising contacting a sample with a dual labeled nucleic acid probe under conditions and for a time sufficient to allow hybridization to a microRNA, wherein the dual labeled nucleic acid probe comprises at least two distinct detectable labels, thereby allowing a substantially double stranded hybrid (or duplex) to form between the microRNA and the nucleic acid probe, contacting the sample with a single stranded nuclease under conditions and for a time sufficient to cleave single stranded regions within the hybrid, and detecting binding of the nucleic acid probe to a single microRNA as coincident signals from the distinct detectable labels. Coincident signals are indicative of a microRNA.

In one embodiment, the nucleic acid probe has a length that is at least 2, at least 3, at least 4, or at least 5 bases longer than the microRNA.

In one embodiment, the nucleic acid probe is a DNA, PNA, LNA or a combination thereof. In one embodiment, the nucleic acid probe is a molecular beacon. In one embodiment, the nucleic acid probe is 22-28 bases long.

In one embodiment, the double stranded hybrid comprises a one or two base overhang at the 3′ and 5′ end of the miRNA. In one embodiment, the one or two base overhang at the 3′ and 5′ end each comprises a cytosine or a guanosine. In one embodiment, the one or two base overhang at the 3′ and 5′ end each comprises an adenine or a thymidine. In one embodiment, the one or two base overhang at the 3′ and 5′ end each comprises an iso-guanosine or an iso-cytosine.

In one embodiment, the method further comprises isolating the double stranded hybrid from the sample. In one embodiment, the method further comprises column purification prior to detecting coincident binding. In one embodiment, the method further comprises addition of a quencher labeled nucleic acid probe to the sample prior to detecting coincident binding.

In one embodiment, the double stranded hybrid is isolated from the sample by size separation. In one embodiment, the sample comprises a plurality of RNA molecules. In one embodiment, the single stranded nuclease is RNase or S1 nuclease.

In another aspect, the invention provides a method for detecting microRNA in sample comprising contacting a sample with a dual labeled nucleic acid probe under conditions and for a time sufficient to allow hybridization to a microRNA, wherein the dual labeled nucleic acid probe comprises a FRET donor fluorophore and a FRET acceptor fluorophore, thereby allowing a substantially double stranded duplex to form between the microRNA and the nucleic acid probe, contacting the sample with a single stranded nuclease under conditions and for a time sufficient to cleave single stranded nucleic acids including single stranded nucleic acid regions within the hybrid, and detecting binding of the nucleic acid probe to a single microRNA as emission from the FRET acceptor fluorophore following excitation of the FRET donor fluorophore. Emission from the FRET acceptor fluorophore is indicative of a microRNA.

In yet another aspect, the invention provides a method for detecting microRNA in a sample comprising contacting a sample with a universal nucleic acid (or linker) having a first sequence specific for a microRNA conjugated to a second sequence that is a universal sequence, under conditions and for a time sufficient to allow hybridization of the universal nucleic acid to a microRNA, thereby forming a double stranded duplex with a 5′ overhang comprising the universal sequence, synthesizing a nucleic acid tail from the miRNA wherein the tail is complementary to the 5′ overhang, thereby creating a tailed miRNA, separating the tailed miRNA from the universal nucleic acid, contacting the tailed miRNA with a miRNA-specific probe labeled with a first detectable label and a universal sequence-specific probe labeled with a second detectable label, wherein the first and second detectable labels are distinct, and detecting coincident binding of the probes to a single microRNA as coincident signals from the first and second detectable labels. Coincident signals are indicative of a microRNA.

In a related aspect, the invention provides a method for detecting microRNA in a sample comprising contacting a sample with a universal nucleic acid having a first sequence specific for a microRNA conjugated to a second sequence that is a universal sequence, under conditions and for a time sufficient to allow hybridization of the universal nucleic acid to a microRNA, thereby forming a double stranded duplex with a 5′ overhang comprising the universal sequence, synthesizing a nucleic acid tail from the miRNA wherein the tail is complementary to the 5′ overhang, thereby creating a tailed miRNA, separating the tailed miRNA from the universal nucleic acid, contacting the tailed miRNA with a miRNA-specific probe labeled with a first fluorophore and a universal sequence-specific probe labeled with a second fluorophore, wherein the first and second fluorophores are a FRET pair comprised of a FRET donor fluorophore and a FRET acceptor fluorophore, and detecting coincident binding of the probes to a single microRNA as emission from the FRET acceptor fluorophore following excitation of the FRET donor fluorophore. Coincident binding is indicative of a microRNA.

In one embodiment, first and second fluorophores are located at proximal ends of the probes when hybridized to the tailed miRNA.

In one embodiment, the universal nucleic acid is at least 20 or at least 40 bases in length.

In one embodiment, each of the probes is independently a DNA, PNA, LNA or a combination thereof.

In one embodiment, the method further comprises isolating the tailed miRNA with coincidentally bound probes from the sample.

In one embodiment, the tailed miRNA with coincidentally bound probes is isolated from the sample by size separation.

In one embodiment, the sample comprises a plurality of RNA molecules. In one embodiment, the method further comprises column purification prior to detecting coincident binding or coincident signals.

In one embodiment, the method further comprises addition of a quencher labeled nucleic acid probe to the sample prior to detecting coincident binding. In one embodiment, the method further comprises ligating the probes to each other prior to detecting coincident binding.

In one embodiment, the detectable labels are located at distal ends of the probes when hybridized to the tailed miRNA.

In another aspect, the invention provides a method for detecting microRNA comprising contacting a sample with a microRNA-specific nucleic acid probe that is conjugated to a first detectable label under conditions and for a time sufficient for specific hybridization of the probe to a microRNA, thereby forming a double stranded duplex and a 5′ overhang comprising microRNA sequence, synthesizing a nucleic acid tail from the microRNA-specific probe wherein the tail is complementary to a 5′ region of the microRNA using nucleotides that are labeled with a second detectable label that is distinct from the first detectable label, thereby forming a dual labeled microRNA-specific probe hybridized to a microRNA, removing single stranded nucleic acids from the sample, and detecting coincident signals from the first and the second detectable labels. Coincident signals are indicative of a microRNA.

In a related aspect, the invention provides a method for detecting microRNA comprising contacting a sample with a microRNA-specific nucleic acid probe that is conjugated to a first fluorophore under conditions and for a time sufficient for specific hybridization of the probe to a microRNA, thereby forming a double stranded duplex and a 5′ overhang comprising microRNA sequence, synthesizing a nucleic acid tail from the microRNA-specific probe wherein the tail is complementary to a 5′ region of the microRNA using nucleotides that are labeled with a second fluorophore, wherein the first and second fluorophores are a FRET pair comprised of a FRET donor and a FRET acceptor fluorophore, thereby forming a dual labeled microRNA-specific probe hybridized to a microRNA, removing single stranded nucleic acids from the sample, and detecting emission from the FRET acceptor fluorophore following excitation of the FRET donor fluorophore. Emission from the FRET acceptor fluorophore is indicative of a microRNA.

In one embodiment, the single stranded nucleic acids are removed from the sample by column purification prior to detecting coincident signals. In one embodiment, the single stranded nucleic acids are removed from the sample by addition of a single stranded nuclease to the sample prior to detecting coincident signals.

In one embodiment, the microRNA-specific probe is at least 2, at least 3, at least 4, at least 5, at least 6, at least 6 or at least 7 bases shorter than the microRNA. In one embodiment, the microRNA-specific probes is at least 15 or at least 20 bases long. In one embodiment, wherein the microRNA-specific probe is a DNA, PNA, LNA or a combination thereof.

In one embodiment, the method further comprises isolating the dual labeled microRNA-specific probe hybridized to a microRNA from the sample.

In one embodiment, the dual labeled microRNA-specific probe hybridized to a microRNA is isolated from the sample by size separation.

In one embodiment, the sample comprises a plurality of RNA molecules.

In one embodiment, the method further comprises addition of a quencher labeled nucleic acid probe to the sample prior to detecting coincident signals.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE FIGURES

FIG. 1 is a schematic of a Direct™ miRNA assay.

FIG. 2 is a graph showing the sensitivity and linear dynamic range of the Direct™ miRNA assay. The inset graph shows the linear range at the lower fM concentration range.

FIG. 3A is a table showing the results of a mir-16 analysis using the Direct™ miRNA assay.

FIG. 3B is a table showing the results from three separate determinations of mir-16 level in various tissues.

FIG. 3C is a graph showing a calibration curve of mir-16.

FIG. 3D is a bar graph showing the expression profile of mir-16 in human total RNA in various tissues.

FIG. 4A is a table showing the results of two separate determinations of mir-126 levels in various human tissues. The assay is highly reproducible even when conducted by different operators on different instruments.

FIG. 4B is a table showing the results of two separate determinations of mir-191 levels in brain.

FIG. 4C is a table showing the results of two separate determinations of mir-136 levels in cervix.

FIG. 4D is a table showing the results of two separate determinations of mir-28 levels in cervix.

FIG. 4E is a table showing the results of two separate determinations of mir-195 levels in thymus and lymph.

FIG. 5A is a bar graph showing levels of mir-143 expression in human cancerous and normal tissues.

FIG. 5B is a bar graph showing levels of mir-145 expression in human cancerous and normal tissues.

FIG. 6A is a schematic of an miRNA assay using DNA or RNA probes.

FIG. 6B is a schematic of an miRNA assay using DNA or RNA probes and a DNase/RNase clean-up step.

FIG. 6C is a schematic of an miRNA assay using DNA or RNA probes and a DNase/RNase protection step.

FIG. 7A is a schematic of an miRNA assay using primer extension.

FIG. 7B is a schematic of an miRNA assay using primer extension and an enzyme clean-up step.

FIG. 8 is a schematic of an miRNA assay using DNA or RNA probes and a ligase.

FIG. 9A is a schematic of an miRNA assay using DNA or RNA probes with a universal nucleic acid.

FIG. 9B is a schematic of an miRNA assay using miRNA assay using modified primer extension and a universal nucleic acid.

FIG. 9C is a schematic of an miRNA assay using a modified labeled primer extension and a universal nucleic acid.

FIG. 10 is a schematic of an miRNA assay using a universal nucleic acid and molecular beacons as probes.

FIG. 11A is a schematic of an miRNA assay using RT extension followed by dual probe hybridization (coincident signal detection).

FIG. 11B is a schematic of an miRNA assay using RT extension followed by dual probe hybridization (FRET).

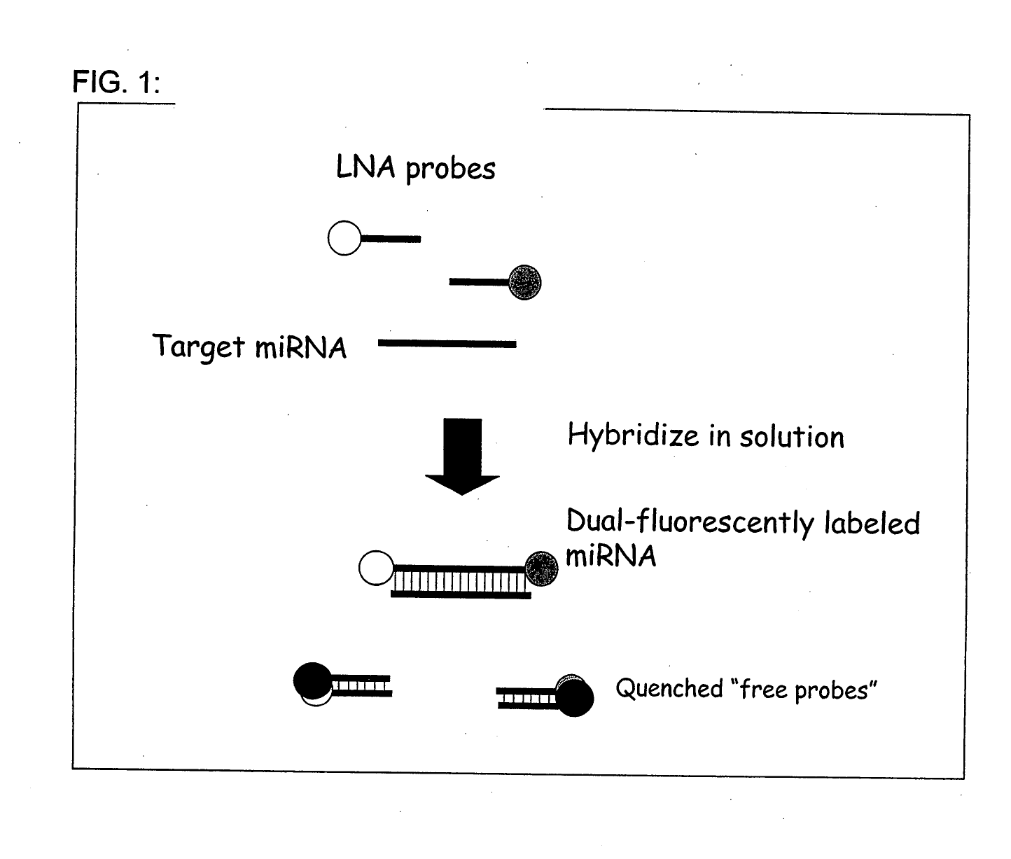

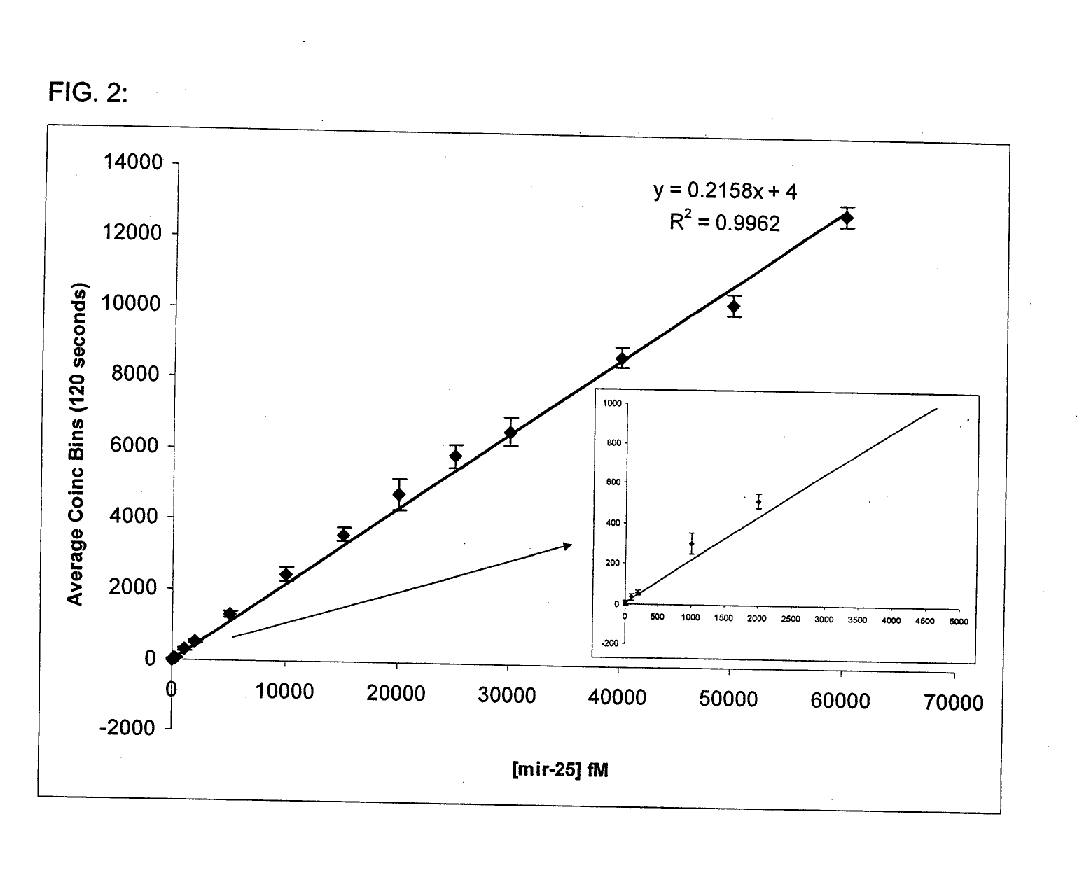

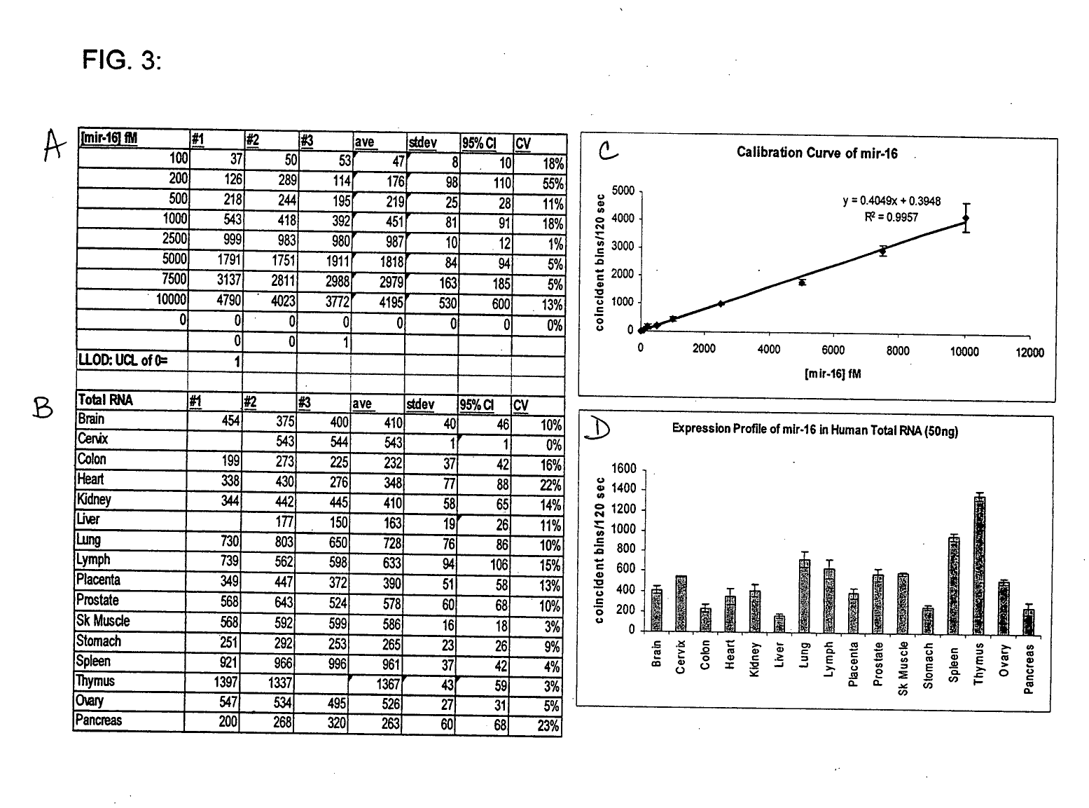

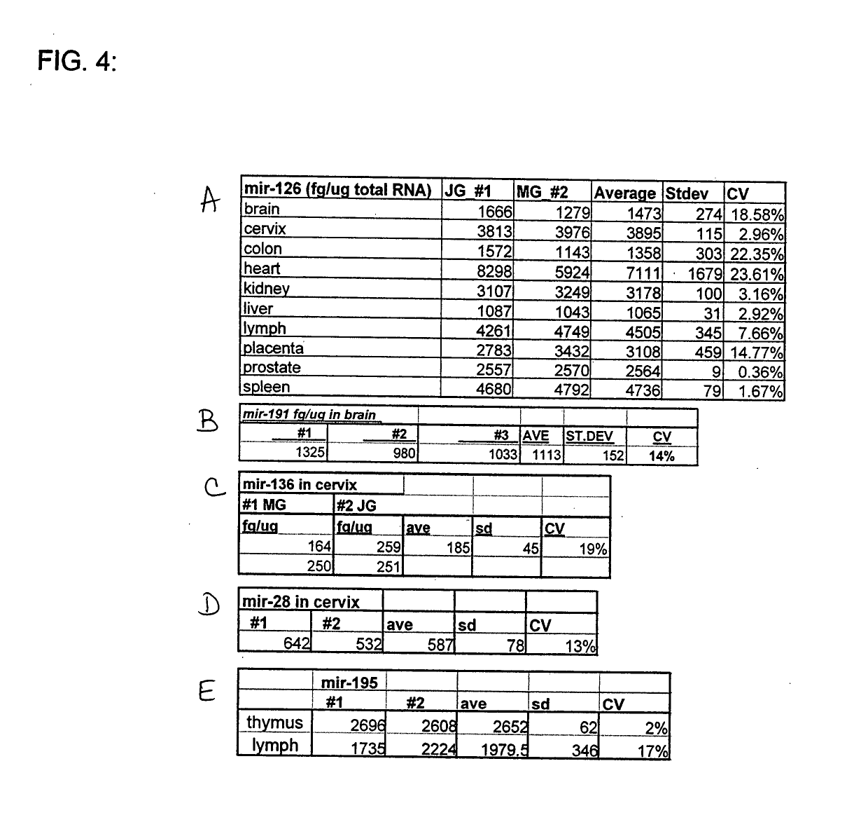

TABLE 1 shows human miRNA expression levels in various tissues.

TABLE 2 shows mir-145 expression levels in tumor, normal adjacent tissues (NAT) and normal tissues.

TABLE 3 shows human miRNA expression levels in bladder and lung.

It is to be understood that the Figures are not required for enablement of the invention.

### BRIEF DESCRIPTION OF THE SEQUENCE LISTING

SEQ ID NOs: 1-34 are nucleotide sequences of a number of human miRNA, as shown herein.

### DETAILED DESCRIPTION OF THE INVENTION

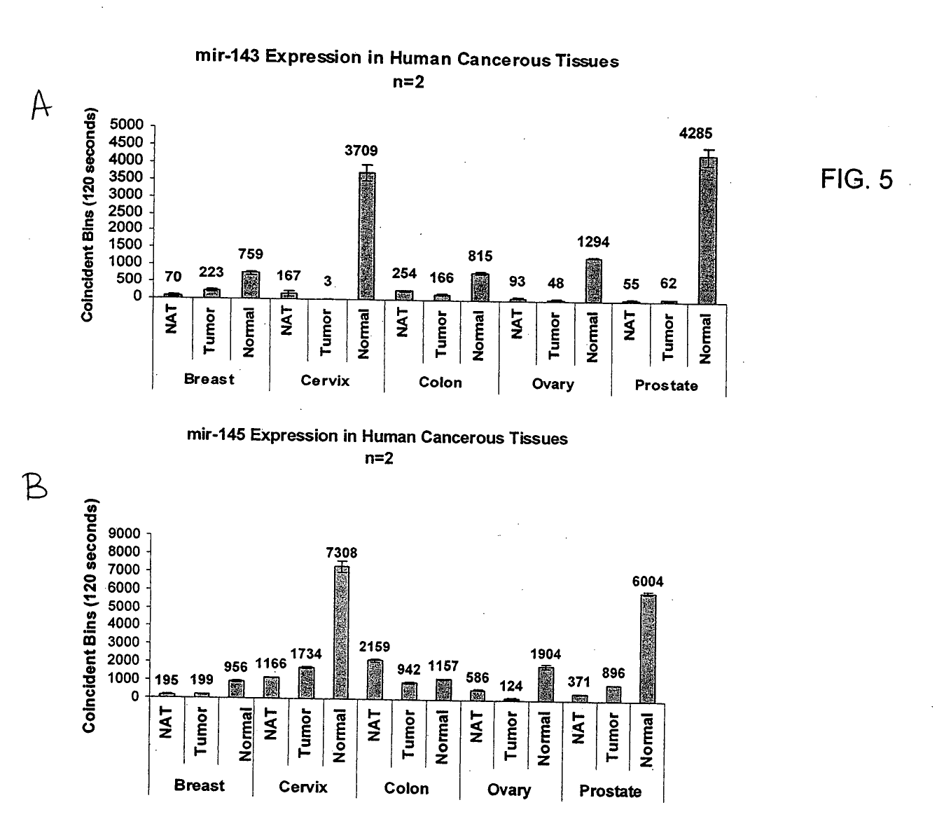

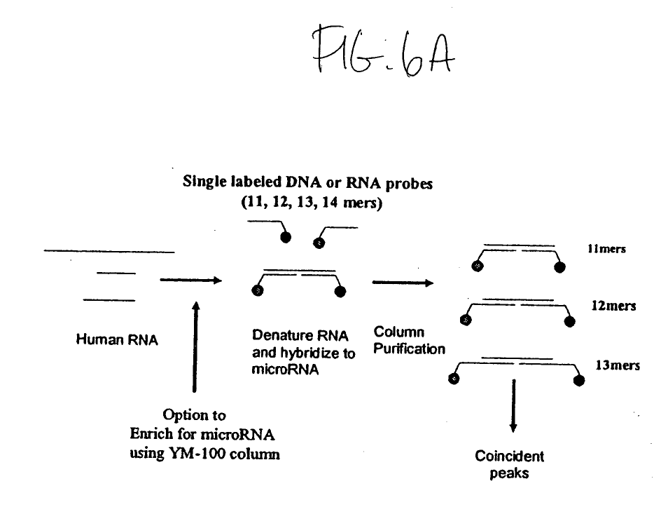

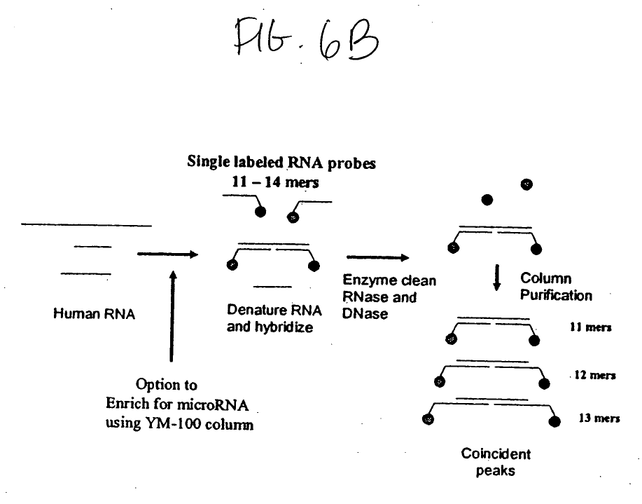

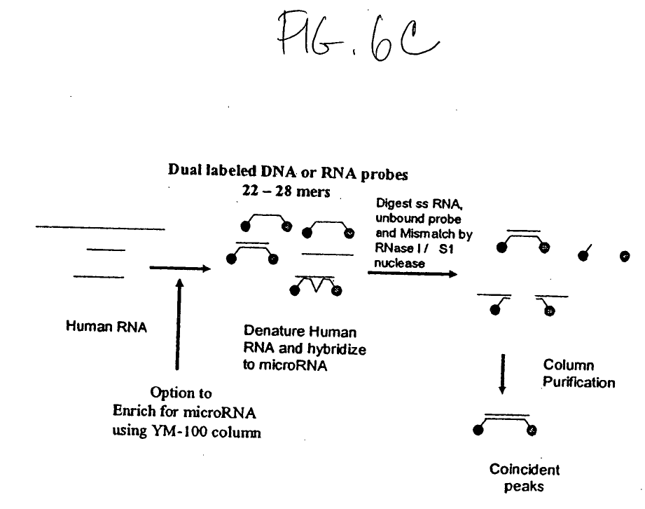

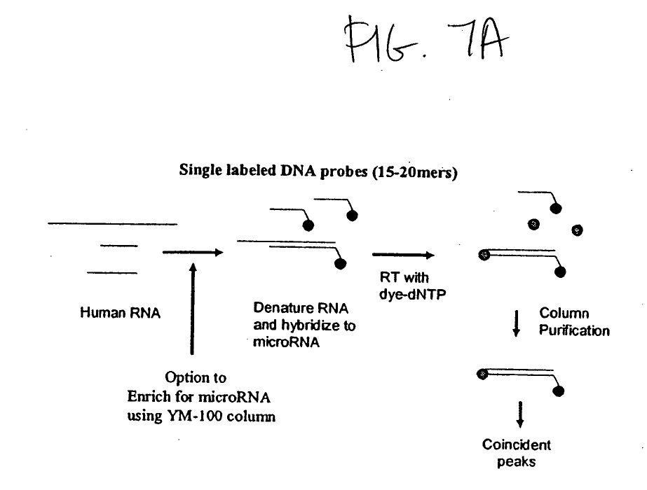

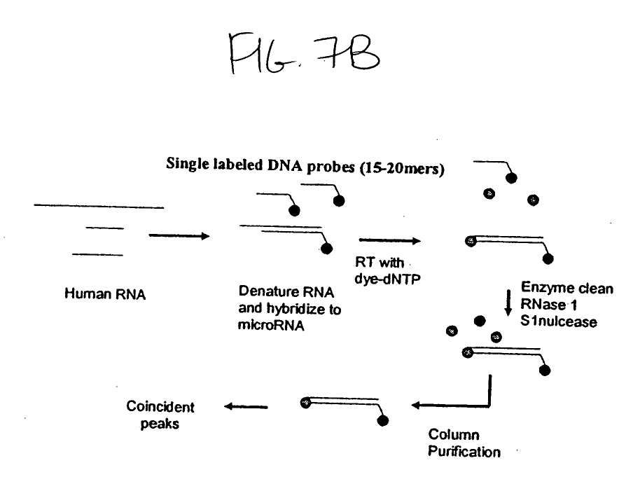

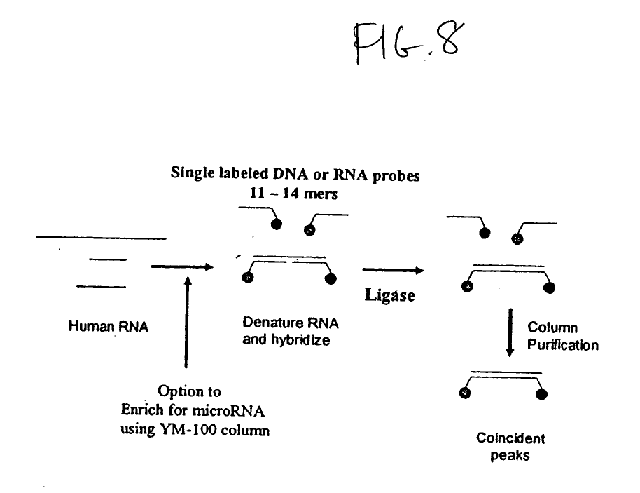

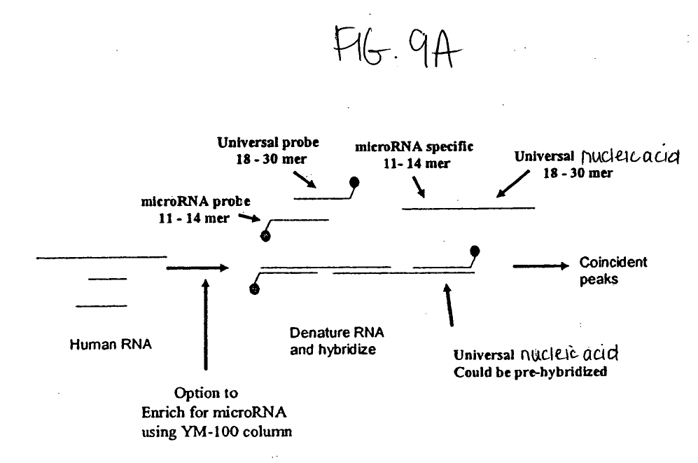

The invention provides inter alia a solution based hybridization assay referred to herein as “Direct™ miRNA” (see FIG. 5). The Direct™ miRNA assay utilizes in some embodiments two spectrally distinguishable probes to label small RNAs of interest. In one working example, a first probe derivatized with Oyster 556 and a second LNA probe derivatized with Oyster 656 have been used. As stated herein, the probes may be comprised of DNA, RNA, PNA, LNA, and the like, or some combination thereof. To conduct the assay, both LNA probes are incubated in molar excess in a hybridization reaction with tissue total RNA. Following hybridization, probe complementary DNA quencher oligonucleotides are added and allowed to hybridize to the unbound fluorescent LNA probes. The reactions are then diluted and subjected to single molecule analysis. Using a method previously described by others (Brinkmeier, 1999) cross-correlation between the two red channels is used to monitor the flow velocity of the fluorescently labeled molecules to ensure even sampling rates across the entire 96-well plate. No enrichment, ligation, reverse transcription, amplification, or clean-up steps are required. The number of coincident photon emissions above a pre-established threshold are counted. This data analysis method provides an estimate of the number of random coincidences expected on the basis of the raw data. This estimate is subtracted from the raw coincidence count to give an estimate of the number of coincidences caused by dual-tagged molecules, and by inference, of the concentration of the analyte.

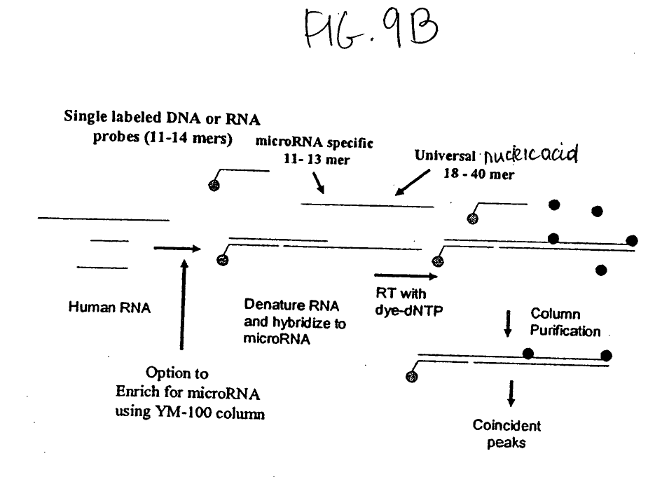

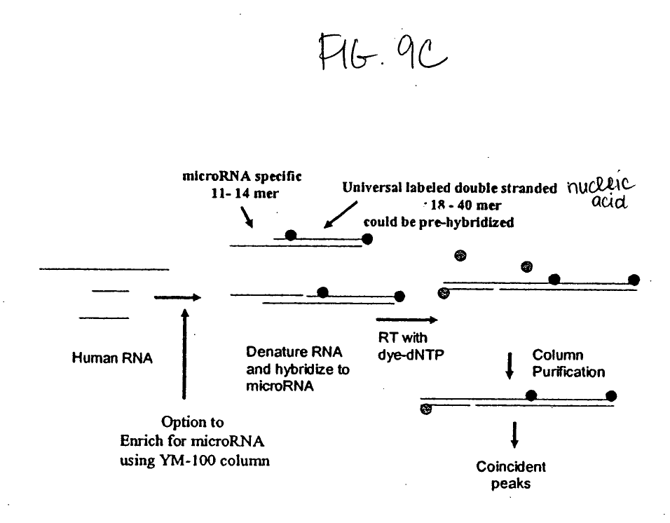

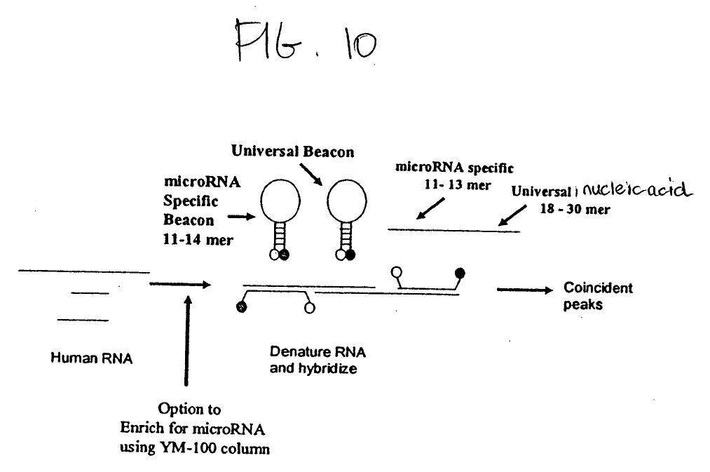

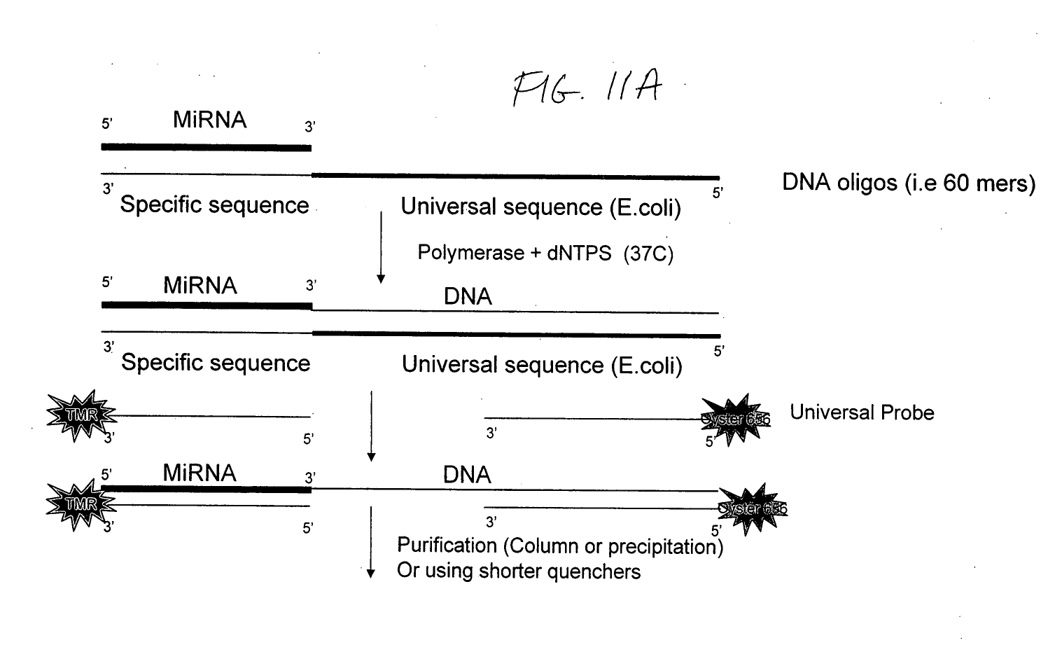

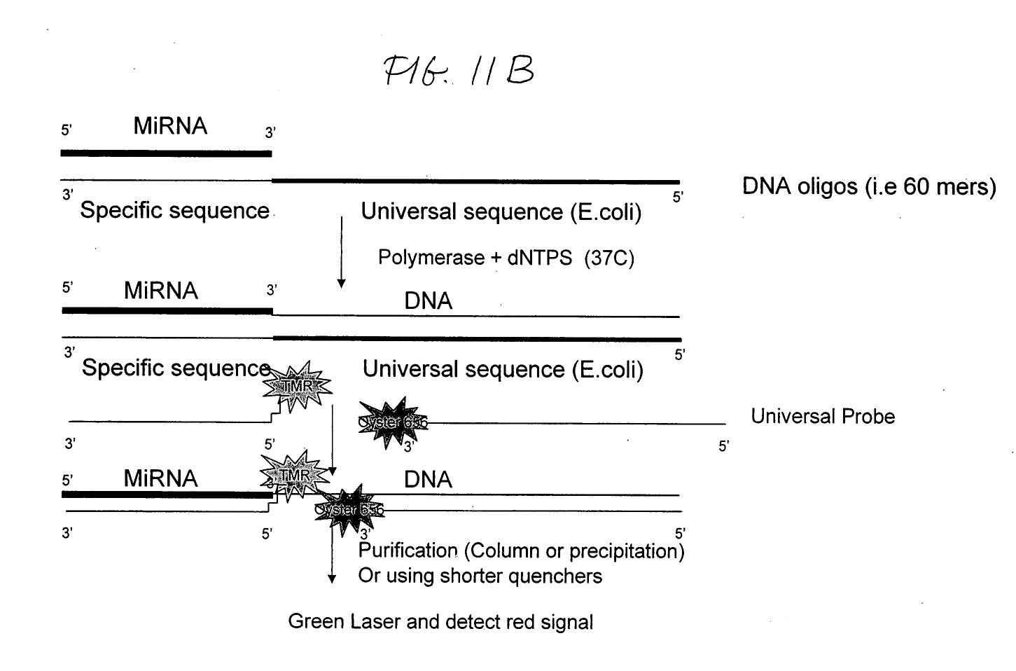

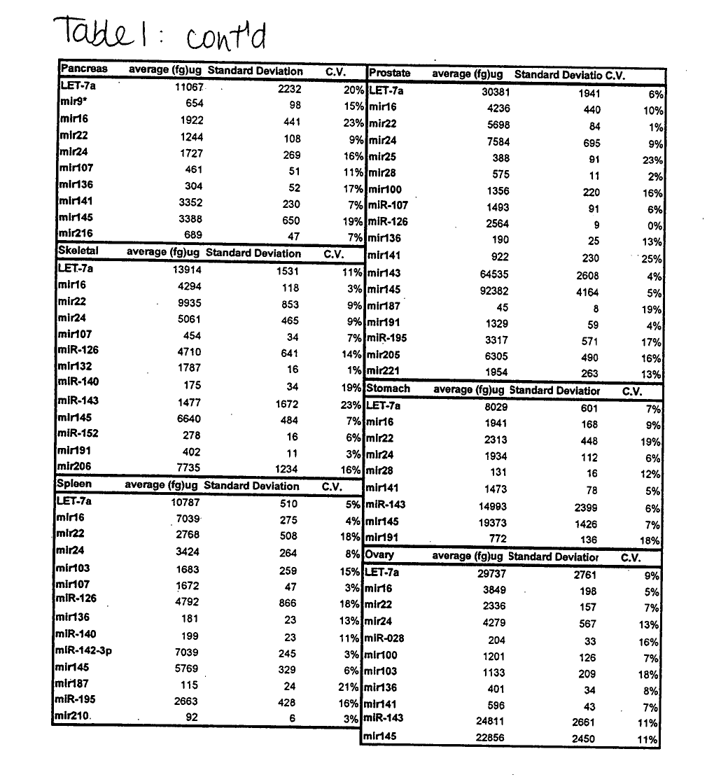

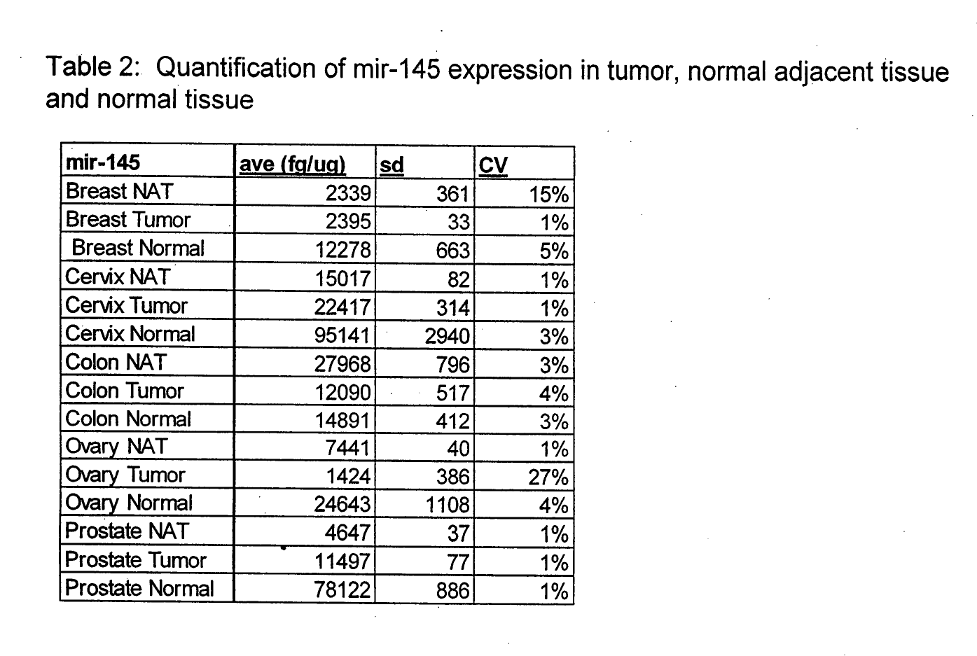

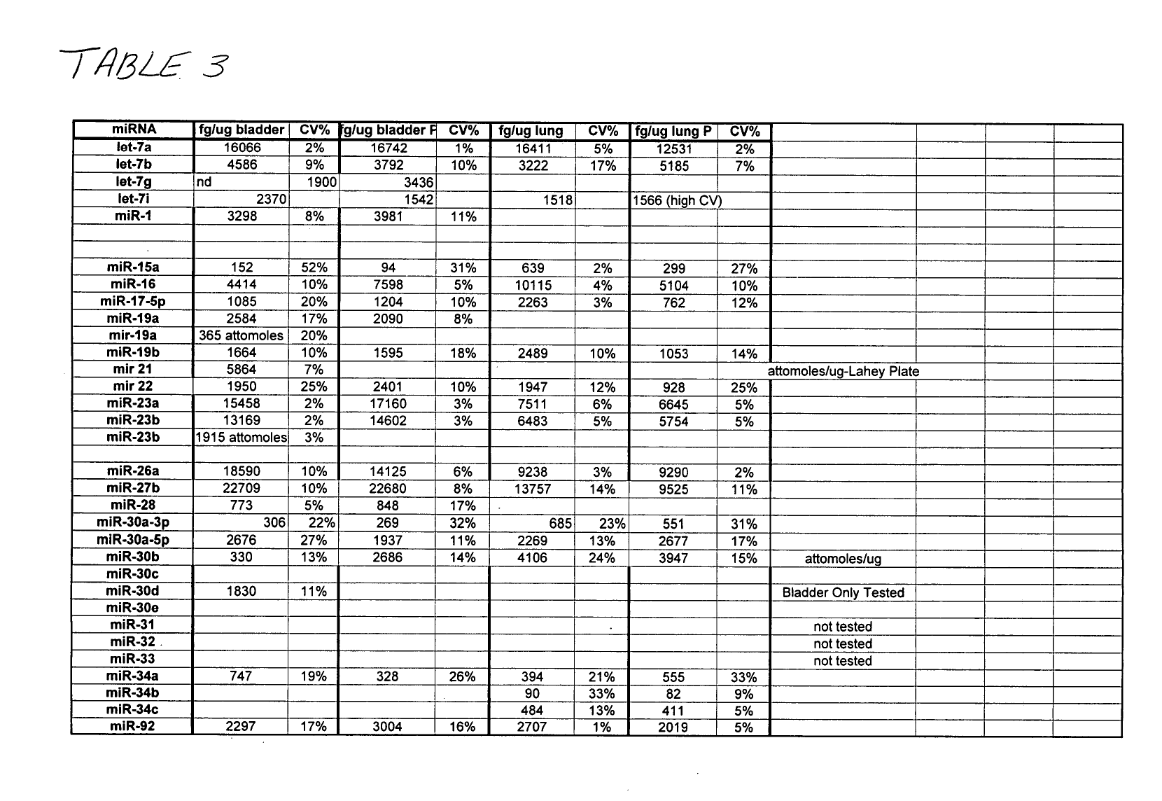

To directly quantify miRNAs within tissue total RNA, three independent calibration curves are prepared by hybridizing known concentrations of a synthetic miRNA target spiked into a complex RNA background. The number of coincident events per two minute sample runs are detected. The data are plotted in a scattergram as concentration versus coincident events detected/120 sec. Ordinary least squares fit of a linear regression model indicates the number of coincident events detected strongly correlates with sample concentration. The coefficients of determination measured in all assays conducted thus far are 0.98 to 0.99. An equation that defines the line is used to calculate the concentration of miRNAs within a complex total RNA sample (see FIG. 6). The assay is sensitive to fM concentrations of miRNA and capable of direct quantification over a 3-log linear dynamic range (see FIG. 7). miRNAs can be quantified accurately and reproducibly (CV<25% for n=2 or 3) (see FIG. 9 and Table 1). This reproducibility was observed with different operators using different experimental platforms, accordingly the assay and platform is a robust means to quantify miRNA gene expression.

The expression of 47 different miRNAs within sixteen human tissues was analyzed. The data shown in Table 1 are presented as femtograms/microgram total RNA but could also be presented as femtomoles miRNA/microgram total RNA, or in terms of concentration. The end result of the assay is a reproducible number that depends on the amount of dual-tagged molecules detected per two minute run.

The quantitative nature of these data makes the method suited for quantification of miRNA expression in disease and normal tissues. The assay and platform has the ability to measure subtle fold changes in expression levels that may be missed using other approaches.

The assay has been further applied to examine the changes in mir-143 and mir-145 expression in adenocarcinoma tumors isolated from cervix, colon, prostate, breast and ovary (see FIG. 9 for detected molecules and Table 2 for femtogram amounts of miRNA) and mir-122a expression in normal and cirrhotic liver total RNA. The results suggest that miRNA expression is reduced in tumor tissue; however a reduction in miRNA expression in the “normal adjacent tissue” total RNA (commonly used as a control to ascertain the fold change observed in the diseased tissue) was also observed as compared to cancer-free tissues. This is the first assay platform to characterize disease progression by directly quantifying the miRNA of interest. Accordingly, the method provided herein is referred to as a direct detection method. A direct detection method is one that minimally does not require pre-amplification (e.g., via PCR) of the target miRNA prior to detection.

The method may also be performed using single miRNA specific probes in some embodiments.

The simplicity, sensitivity, rapidity and reproducibility of the assay and its detection platform represent a significant advance in the quantification of miRNA expression. The instrument and assay are also completely automatable and as such a superior means for identifying and characterizing disease in a diagnostic setting. The power of a quantifiable number (e.g., femtograms miRNA) will facilitate detection and fine characterization of disease states and progression and thereby lead to significant advances in disease treatment.

The method embraces the establishment of databases that contain miRNA expression level data for a variety of normal and abnormal tissue types, and comparison of miRNA expression data from test tissue samples to such databases. Accordingly the miRNA expression levels from a test tissue sample can be compared to a normal control from the same or a different subject prepared concurrently with (or prior to) the test tissue sample, or to expression levels previously determined for one or more abnormal (and optionally normal) tissue types.

It should be understood that the method further provides the ability to profile conditions or disease states based on expression (or lack thereof) of one or more miRNA. This allows a more accurate characterization of a disease state and its associated prognosis.

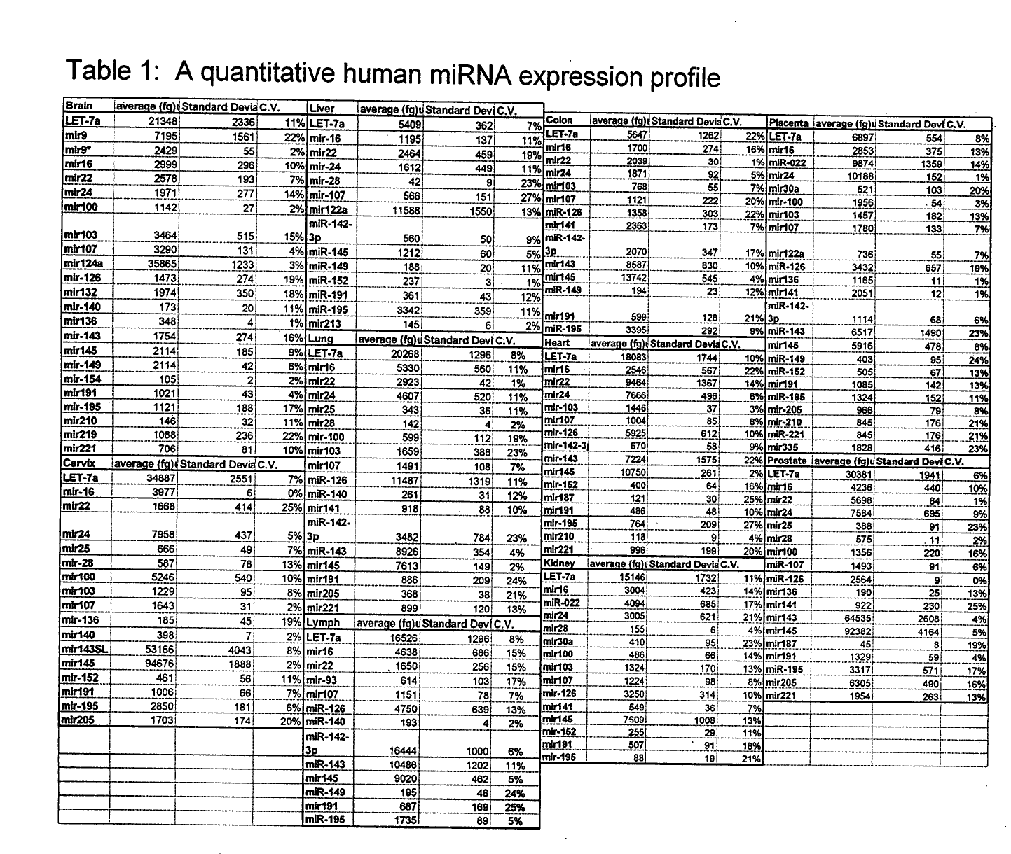

Other aspects of the invention relate to detection of miRNA. In one aspect, a method is provided for detecting a miRNA in a sample that comprises contacting a sample with a first and a second probe wherein the first probe comprises a first detectable label and the second probe comprises a second detectable label, wherein the first and second detectable labels are distinct from each other, for a time and under conditions that allow binding of the first and second probes to their respective targets, and detecting a single miRNA that is bound to both the first and the second probes by coincident detection of the first and second detectable labels. (See FIGS. 6A-11B.)

The probes may be of any length. Their combined length may be equal to or greater than the length of the miRNA target. For example, if the miRNA target is 22 bases in length, the probes may each be 11, 12, 13 or 14 bases in length. In some embodiments, the probes are of such a length that once bound to the target there exists a one or two or more base overhang at both ends of the duplex.

Detection of the single miRNA may be preceded by a “clean-up” step. Such intervening steps are generally intended to separate unreacted reagents (such as unbound probes) from duplexes comprising the target and two probes bound thereto. The clean up step may comprise use of a column that separates hybridization reaction components according to size. Another clean up approach may comprise use of enzymes such as RNase and/or DNases to digest single stranded probes (which can be RNA or DNA in nature) as well as unbound targets.

In still other embodiments, the two bound probes may be ligated to each other through the action of a ligase, thereby resulting in a double stranded duplex at least 20 or 22 base pairs in length.

In another aspect, miRNA is detected using a dual labeled probe (FIG. 6C). As used herein, a dual labeled probe is a probe comprising two distinct labels, preferably, one at each end of the probe. The probes may be DNA or RNA in nature, and their lengths may range from 22-28 bases in some embodiments. Once the probes are allowed to bind to their targets, the sample is contacted with a DNase or an RNase that recognizes and nicks single stranded mismatches. The nuclease therefore would digest unbound probe as well as duplexes having single stranded mismatches. The method would further comprise detection of a single miRNA target having a dual labeled probe bound thereto.

In still another aspect, miRNA is detected using primer extension (FIGS. 7A, 7B, 9B, 9C, 11A and 11B). The method comprises contacting miRNA with single stranded DNA probes that are labeled with a first detectable label, and then performing a reverse transcription (RT) reaction to extend the probe in the presence of nucleotides that are labeled with a second detectable label that is distinct from the first label. Single duplexes comprising the target miRNA and the dually labeled extended probe are then detected and are indicative of the presence of the miRNA in the sample. Alternatively, the dually labeled extended probe may also be detected independent of its hybridization to the target miRNA. The dually labeled extended probe is detected via coincident detection of signals from both labels. It will be understood by one of ordinary skill in the art that the probes in the afore-mentioned method function as primers for the RT reaction. As such, the probes may be of any length that is less than the length of the target miRNA. For example, the probes may be 5-20 nucleotides in length. It is to be understood that a pool of labeled nucleotides may be used in the RT reaction, wherein the pool contains a first subset of nucleotides labeled with a second detectable label, a second subset of nucleotides labeled with a third detectable label, etc. provided that the each of the detectable labels is distinct from every other detectable label used in the reaction. As with previous methods, the RT reaction mixture may be manipulated in order to remove unreacted substrates such as unincorporated labeled nucleotides. This may be accomplished using a column purification step or a single stranded nuclease (e.g., RNase or S1 nuclease) digestion, or a combination of both, but it is not so limited.

Some detection methods may comprise the use of a universal nucleic acid having one sequence that is miRNA specific and a second region that comprises a universal sequence and therefore that acts as a universal linker (FIGS. 9A, 9B, 9C, 10, 11A and 11B). A universal sequence is a sequence of known composition that may be common amongst a plurality of universal nucleic acids. Thus, in one aspect, the invention provides a method in which single-stranded DNA or RNA probes are contacted with a sample, and allowed to hybridize to their respective target miRNA. The probes are shorter than the target miRNA. They may range in size from 11-14 nucleotides, but they are not so limited. The universal nucleic acid may be included in the initial hybridization reaction or it may be included in a subsequent hybridization reaction. The universal nucleic acid binds to the single stranded region of the target miRNA (i.e., the region not hybridized to the labeled probe). In doing so, a duplex is formed comprising the labeled probe, the target miRNA, and the universal nucleic acid. This duplex further contains a single stranded region that is available as a template for an RT reaction that is primed from the miRNA. The RT reaction is carried out in the presence of labeled nucleotides, as described above. The method further comprises detecting a duplex comprising the labeled probe, the universal nucleic acid, and the labeled and extended target miRNA. Thus, in this embodiment, the labels are present on opposite strands of the duplex and thus coincident detection of distinct signals requires that the duplex remains intact. A column purification step may be carried out prior to coincident detection analysis.

In another aspect, another method is provided for detecting miRNA that comprises a partially double stranded universal nucleic acid (FIG. 9C). The linker may be pre-hybridized prior to further manipulation. The universal nucleic acid comprises a first strand that is detectably labeled and a second strand that is not detectably labeled. The second strand is longer than the first strand, thereby creating a single stranded unlabeled region. This region is specific for a target miRNA. When contact with a sample containing the target miRNA occurs, the double stranded universal nucleic acid binds to its respective target miRNA resulting in a new single stranded overhang that then serves as a template for extending the second strand of the universal nucleic acid. This is accomplished by performing an RT reaction with labeled nucleotides. This leads to the formation of a duplex comprising the double stranded universal nucleic acid with its second strand extended and now labeled and the target miRNA. The two strands of the duplex are distinctly labeled from one and other. The method then further comprises detection of the duplex based on coincident detection of signals from the at least two different detectable labels.

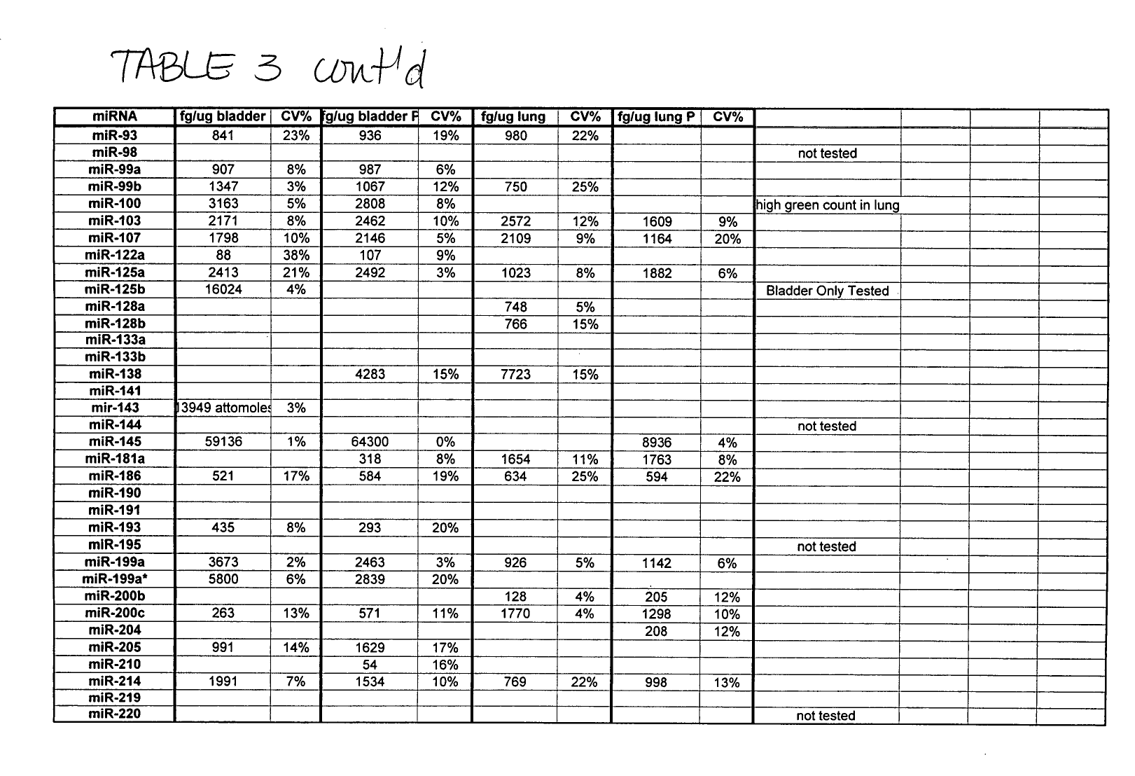

In still another aspect, a method for detecting miRNA is provided that uses both a universal nucleic acid and at least two probes that are molecular beacons (FIG. 10). A first probe is specific for the target miRNA. A second probe is specific for the universal nucleic acid. Each probe further comprises a detectable label, the signal from which is quenched (by a quencher moiety) when the probe is not bound to its target. The detectable labels on the first and second probes are distinct from each other. Upon hybridizing with its target (whether the miRNA or the universal nucleic acid), the stem hybridization of the probe is denatured (or melted) and the quencher moiety is no longer in close enough proximity to the detectable label to effectively quench the latter's signal. The universal nucleic acid comprises an miRNA specific sequence (that may be for example 11-13 nucleotides in length) and a universal sequence (that may be for example 18-30 nucleotides in length). When contacted and allowed to hybridize, the first probe will bind to the target miRNA, as will the universal nucleic acid. The universal nucleic acid in turn will also bind to the second probe. This will result in a double stranded complex comprising both probes (with each emitting its own distinct signal), the target miRNA, and the universal nucleic acid. Single complexes will then be detected via coincident detection of signals from both probes.

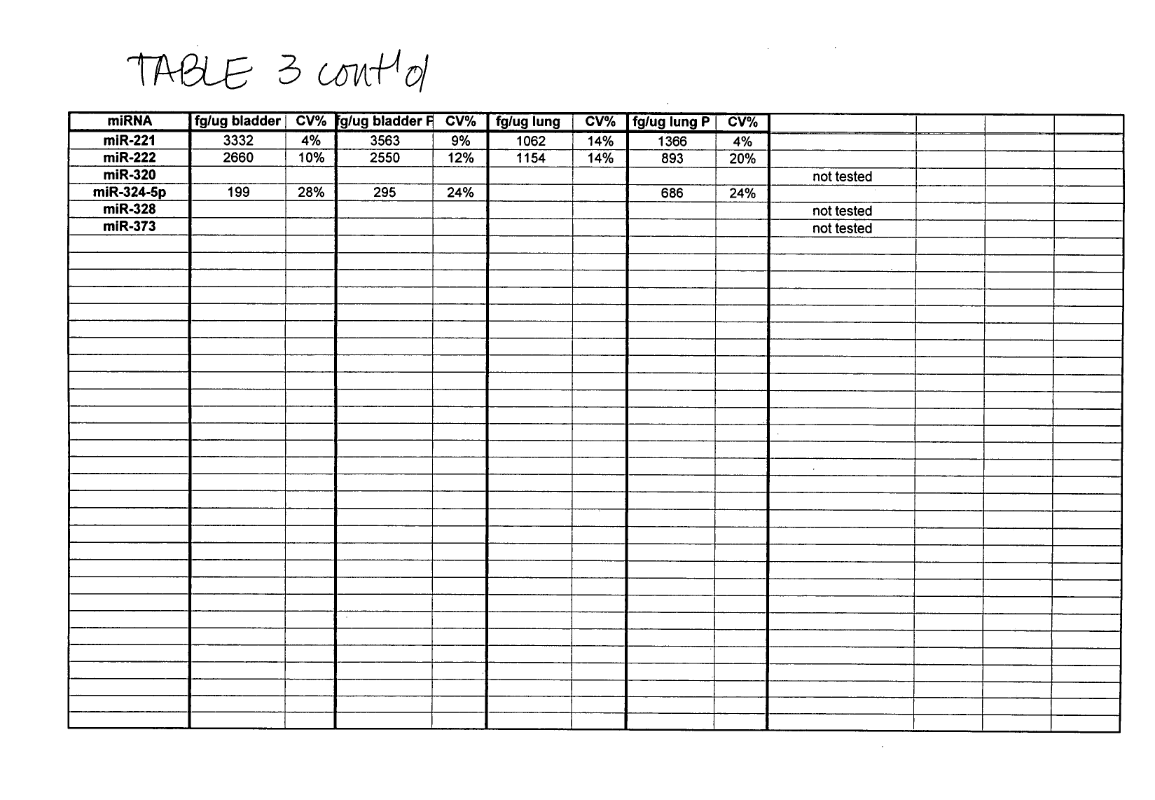

In still another aspect, a method is provided for detecting miRNA by contacting a sample comprising miRNA with a universal nucleic acid (FIG. 11A). The universal nucleic acid comprises an miRNA specific sequence and a universal sequence. The universal nucleic acid is allowed to hybridize to its target miRNA, and the resulting complex comprises a double stranded region and a single stranded region. The target miRNA acts as a primer that is extended using an RT reaction in the presence of nucleotides. The resulting double stranded complex is then denatured and contacted with a first probe that is labeled with a first detectable label and a second probe that is labeled with a second detectable label that is distinct from the first label. For example, one label may be Tamra while the other may be Oyster 656. One probe is therefore specific for the miRNA sequence while the other probe is specific for the universal sequence. Single complexes comprising the extended miRNA strand hybridized to both probes are then detected via coincident detection of signals from both labels. As with afore-mentioned methods, column separation, precipitation, and/or quencher conjugated probes may be used in order to remove unreacted substrates. The universal nucleic acid may be any length that is greater than the target miRNA. For example, it may be 30, 40, 50, 60, or more nucleotides in length. The universal sequence should be chosen so as not to interfere with hybridization to the target miRNA. For example, sequence from E. coli may be used.

In a variation of the latter method, the probes are each labeled at their opposite end such that, when hybridized to the extended miRNA, the labels are in sufficient proximity to undergo FRET (FIG. 11B). In this embodiment, the coincident binding of the probes is detected via detection of signal from one of the labels upon excitation of the other label. For example, one probe may be labeled with Tamra and the other may be labeled with Oyster 656, and a green laser is used in order to detect a red signal.

In any of the foregoing aspects, the sample may be a sample that is harvested in accordance with RNA isolation methods. In some embodiments, miRNA may be enriched using a YM-100 column.

miRNA

miRNA is a short non-coding RNA molecule, usually about 22 nucleotides in length. The sequences of numerous miRNA are known and publicly available. Accordingly, synthesis of miRNA-specific probes is within the ordinary skill in the art based on this information. miRNA sequences can be accessed at for example the website of the miRNA Registry of the Sanger Institute (Wellcome Trust), or the website of Ambion, Inc.

For example, some miRNA sequences are as follows:

| | | | SEQ |
|---|---|---|---|
| | | Accession | ID |
| miRNA | Sequence | Number | NO: |
| human mir-9 | UCUUUGGUUAUCUAGCUGUAUGA | MI0000466 | 1 |
| human mir-16 | UAGCAGCACGUAAAUAUUGGCG | MI0000738 | 2 |
| human mir-22 | AAGCUGCCAGUUGAAGAACUGU | MI0000078 | 3 |
| human mir-24 | GUGCCUACUGAGCUGAUAUCAGU | MI0000080 | 4 |
| human mir-25 | CAUUGCACUUGUCUCGGUCUGA | MI0000082 | 5 |
| human mir-28 | AAGGAGCUCACAGUCUAUUGAG | MI0000086 | 6 |
| human mir-30a | UGUAAACAUCCUCGACUGGAAG | MI0000088 | 7 |
| human mir-93 | AAAGUGCUGUUCGUGCAGGUAG | MI0000095 | 8 |
| human mir-100 | AACCCGUAGAUCCGAACUUGUG | MI0000102 | 9 |
| human mir-103 | AGCAGCAUUGUACAGGGCUAUGA | MI0000109 | 10 |
| human mir-107 | AGCAGCAUUGUACAGGGCUAUCA | MI0000114 | 11 |
| human mir-122a | UGGAGUGUGACAAUGGUGUUUGU | MI0000442 | 12 |
| human mir-124a | UUAAGGCACGCGGUGAAUGCCA | MI0000443 | 13 |
| human mir-126 | CAUUAUUACUUUUGGUACGCG | MI0000471 | 14 |
| human mir-132 | UAACAGUCUACAGCCAUGGUCG | MI0000449 | 15 |
| human mir-136 | ACUCCAUUUGUUUUGAUGAUGGA | MI0000475 | 16 |
| human mir-140 | AGUGGUUUUACCCUAUGGUAG | MI0000456 | 17 |
| human mir-141 | CAUAAAGUAGAAAGCACUAC | MI0000458 | 18 |
| human mir-142 | CAUAAAGUAGAAAGCACUAC | MI0000458 | 19 |
| human mir-143 | UGAGAUGAAGCACUGUAGCUCA | MI0000459 | 20 |
| human mir-145 | GUCCAGUUUUCCCAGGAAUCCCUU | MI0000461 | 21 |
| human mir-149 | UCUGGCUCCGUGUCUUCACUCC | MI0000478 | 22 |
| human mir-152 | UCAGUGCAUGACAGAACUUGGG | MI0000462 | 23 |
| human mir-154 | UAGGUUAUCCGUGUUGCCUUCG | MI0000480 | 24 |
| human mir-187 | UCGUGUCUUGUGUUGCAGCCG | MI0000274 | 25 |
| human mir-191 | CAACGGAAUCCCAAAAGCAGCU | MI0000465 | 26 |
| human mir-195 | UAGCAGCACAGAAAUAUUGGC | MI0000489 | 27 |
| human mir-205 | UCCUUCAUUCCACCGGAGUCUG | MI0000285 | 28 |
| human mir-206 | UGGAAUGUAAGGAAGUGUGUGG | MI0000490 | 29 |
| human mir-210 | CUGUGCGUGUGACAGCGGCUGA | MI0000286 | 30 |
| human mir-213 | CAUAAAGUAGAAAGCACUAC | MI0000458 | 31 |
| human mir-216 | UAAUCUCAGCUGGCAACUGUG | MI0000292 | 32 |
| human mir-219 | UGAUUGUCCAAACGCAAUUCU | MI0000296 | 33 |
| human mir-221 | AGCUACAUUGUCUGCUGGGUUUC | MI0000298 | 34 |

Sample

Harvest and isolation of total RNA is known in the art and reference can be made to standard RNA isolation protocols. (See, for example, Maniatis' Handbook of Molecular Biology.) The method does not require that miRNA be enriched from a standard RNA preparation. However, if desired, miRNA can be enriched using, for example, a YM-100 column.

The methods of the invention may be performed in the absence of prior nucleic acid amplification in vitro. Preferably, the miRNA is directly harvested and isolated from a biological sample (such as a tissue or a cell culture), without its amplification. Such miRNA are referred to as “non in vitro amplified nucleic acids”. As used herein, a “non in vitro amplified nucleic acid” refers to a nucleic acid that has not been amplified in vitro using techniques such as polymerase chain reaction or recombinant DNA methods.

A non in vitro amplified nucleic acid may, however, be a nucleic acid that is amplified in vivo (e.g., in the biological sample from which it was harvested) as a natural consequence of the development of the cells in the biological sample. This means that the non in vitro nucleic acid may be one which is amplified in vivo as part of gene amplification, which is commonly observed in some cell types as a result of mutation or cancer development.

miRNA to be detected and optionally quantitated are referred to as target miRNA or target nucleic acids.

miRNA may be harvested from a biological sample such as a tissue or a biological fluid. The term “tissue” as used herein refers to both localized and disseminated cell populations including, but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art. In some embodiments, the miRNA are harvested from one or few cells.

The biological sample can be normal or abnormal (e.g., malignant). Malignant tissues and tumors include carcinomas, sarcomas, melanomas and leukemias generally and more specifically biliary tract cancer, bladder cell carcinoma, bone cancer, brain and CNS cancer, breast cancer, cervical cancer, choriocarcinoma, chronic myelogenous leukemia, colon cancer, connective tissue cancer, cutaneous T-cell leukemia, endometrial cancer, esophageal cancer, eye cancer, follicular lymphoma, gastric cancer, hairy cell leukemia, Hodgkin's lymphoma, intraepithelial neoplasms, larynx cancer, lymphomas, liver cancer, lung cancer (e.g. small cell and non-small cell), melanoma, multiple myeloma, neuroblastomas, oral cavity cancer, ovarian cancer, pancreatic cancer, prostate cancer, rectal cancer, renal cell carcinoma, sarcomas, skin cancer, squamous cell carcinoma, testicular cancer, thyroid cancer, and renal cancer. The method may be used to distinguish between benign and malignant tumors.

Subjects from whom such tissue samples may be harvested include those at risk of developing a cancer. A subject at risk of developing a cancer is one who has a high probability of developing cancer (e.g., a probability that is greater than the probability within the general public). These subjects include, for instance, subjects having a genetic abnormality, the presence of which has been demonstrated to have a correlative relation to a likelihood of developing a cancer that is greater than the likelihood of the general public, and subjects exposed to cancer causing agents (i.e., carcinogens) such as tobacco, asbestos, or other chemical toxins, or a subject who has previously been treated for cancer and is in apparent remission.

Some subjects tested may have detectable cancer cells. In these embodiments, the method may be used to more finely characterize the cancer and optionally its stage of development, and thereby optionally provide a prognosis. A subject having a cancer is a subject that has detectable cancerous cells.

Sample Manipulation

Although the miRNA may be linearized or stretched prior to analysis, this is not necessary since the detection system is capable of analyzing both stretched and condensed forms. This is particularly the case with coincident events since these events simply require the presence of at least two labels, but are not necessarily dependent upon the relative positioning of the labels (provided however that if they are being detected using FRET, they are sufficiently proximal to each other to enable energy transfer).

As used herein, stretching of the miRNA means that it is provided in a substantially linear, extended (e.g., denatured) form rather than a compacted, coiled and/or folded (e.g., secondary) form. Stretching the miRNA prior to analysis may be accomplished using particular configurations of, for example, a single molecule detection system, in order to maintain the linear form. These configurations are not required if the target can be analyzed in a compacted form.

The sample or reaction mixture may be cleaned prior to analysis, although the method provided herein does not require such a step. As used herein “cleaning” refers to the process of removing unbound probes. This cleaning step can be accomplished in a number of ways including but not limited to column purification. Column purification generally involves capture of small molecules within a column with flow-through of larger molecules (such as the target miRNA and duplexes containing them). It is to be understood however that the method can be performed without removal of these reagents prior to analysis, particularly since coincident detection can distinguish between desired binding events and artifacts. Thus, in some embodiments, unreacted substrates including unbound detectable probes are not removed prior to analysis.

Another way of cleaning up the sample prior to analysis is through the use of quencher-conjugated probes. A quencher-conjugated probe is a probe that binds specifically to the detectable labeled probe used to analyze the target nucleic acid and comprises a quencher molecule. Quencher molecules are molecules that absorb and thereby quench fluorescence from a sufficiently proximal fluorophore (approx. 10-100 A°). The quencher-fluorophore interaction is essentially a FRET phenomenon with the fluorophore being the donor and the quencher being the acceptor molecule. Generally, quencher-conjugated probes can be designed such that the quencher will be proximal to the fluorophore on the complementary probe. Thus, for example, if the sequence-specific probe has a fluorophore at its 3′ end, then the corresponding complementary quencher-conjugated probe may have the quencher located at its 5′ end, and vice versa. Quencher molecules do not re-emit fluorescence after interacting with a fluorophore. As a result, interaction of unbound fluorescent probes with quencher-conjugated probes is effectively the same as physically removing the unbound probes from the reaction mixture, without the potential for any loss of sample or target nucleic acid.

Quenchers are usually multiple ring structures that dissipate the absorbed fluorescent energy via heat. Examples include Black Hole Quenchers (e.g., BHQ-1, BHQ-2, BHQ-3) from Molecular Probes and BioSearch Technologies (Novato, Calif.), and Iowa Black Quencher from IDT. A variety of quenchers are available such that fluorescence between 480-730 nm can be effectively quenched. The absorption spectra of quenchers can be quite broad and therefore a given quencher may be used to quench multiple fluorophore emissions. For example, BHQ-1 has a maximum absorption wavelength of 534 nm but it can actually absorb emissions from 6-FAM (518 nm), TET (538 nm), HEX (553)/JOE (554) and Cy3 (565 nm), as well as others. BHQ-2 has a maximum absorption wavelength of 579 nm but it can actually absorb emissions from TET, HEX/JOE, Cy3, TAMRA (583 nm) and ROX (607 nm), as well as others. BHQ-3 has a maximum absorption wavelength of 672 nm but it can actually absorb emissions from LC Red (640 nm) and Cy5 (667 nm), as well as others.

Commercial sources of quenchers generally conjugate the quencher to a nucleic acid probe of interest. Alternatively, kits for performing such conjugation are also commercially available.

According to the invention, the quencher-conjugated probes are generally nucleic acid (e.g., DNA) in nature and are thus complementary to the miRNA-specific probes used. They must be sufficiently complementary to the sequence-specific probes used in order to bind to such probes specifically. Probes that bind specifically to the target of interest are probes that demonstrate preferential binding to the target than to any other compound. Specific probes have a higher binding affinity for their targets than for another compound. A higher binding affinity may be at least 2-fold, at least 3-fold, at least 4-fold, at least 5-fold, at least 10-fold, at least 100-fold, or greater. The quencher-conjugated probes are added to a reaction mixture at the same time as or following the sequence-specific probes. The reaction conditions are manipulated in order to ensure that the sequence-specific probes preferably bind to the target nucleic acid (in a sequence-specific manner) and that once all probe target sites are saturated, the unbound probes will bind to the quencher-conjugated probes. The invention contemplates modulation of factors such as temperature, buffer conditions (including pH and salt) and hybridization times in order to accomplish this result.

Coincidence Binding and Detection

Coincident binding refers to the binding of two or more probes on a single molecule or complex. Coincident binding of two or more probes is used as an indicator of the molecule or complex of interest. It is also useful in discriminating against noise in the system and therefore increases the sensitivity and specificity of the system. Coincident binding may take many forms including but not limited to a color coincident event, whereby two colors corresponding to a first and a second detectable label are detected. Coincident binding may also be manifest as the proximal binding of a first detectable label that is a FRET donor fluorophore and a second detectable label that is a FRET acceptor fluorophore. In this latter embodiment, a positive signal is a signal from the FRET acceptor fluorophore upon laser excitation of the FRET donor fluorophore.

The methods provided herein involve the ability to detect single molecules based on the temporally coincident detection of detectable labels specific to the miRNA being analyzed. As used herein, coincident detection refers to the detection of an emission signal from more than one detectable label in a given period of time. Generally, the period of time is short, approximating the period of time necessary to analyze a single molecule. This time period may be on the order of a millisecond. Coincident detection may be manifest as emission signals that overlap partially or completely as a function of time. The co-existence of the emission signals in a given time frame may indicate that two non-interacting molecules, each individually and distinguishably labeled, are present in the interrogation spot at the same time. An example would be the simultaneous presence of two unbound but detectably and distinguishably labeled probes in the interrogation spot. However, because the spot volume is so small (and the corresponding analysis time is so short), the likelihood of this happening is small. Rather it is more likely that if two probes are present in the interrogation spot simultaneously, this is due to the binding of both probes to a single molecule passing through the spot. In some embodiments, signals from samples containing labeled probes but lacking miRNA targets are determined and subtracted from signals from samples containing both probes and targets.

The coincident detection methods of the invention involve the simultaneous detection of more than one emission signal. The number of emission signals that are coincident will depend on the number of distinguishable detectable labels available, the number of probes available, the number of components being detected, the nature of the detection system being used, etc. Generally, at least two emission signals are being detected. In some embodiments, three emission signals are being detected. However, the invention is not so limited. Thus, where multiple components are being detected in a single analysis, 4, 5, 6, 7, 8, 9, 10 or more emission signals can be detected simultaneously.

Coincident detection analysis is able to detect single molecules at very low concentrations. For example, as discussed herein, low femtomolar concentrations can be detected using a two or three emission signal approach. In addition, the analysis demonstrates a dynamic range of greater than four orders of magnitude. A two emission signal approach is also able to detect single molecules such as single proteins at levels below 1 ng/ml.

Multiplexing

Single miRNAs are detected using one or more probes that are specific to the miRNA (i.e., miRNA-specific probes, as discussed herein). A sample may be tested for the presence of a miRNA by contacting it with one or more miRNA-specific probes for a time and under conditions that allow for binding of the probes to the miRNA if it is present. Excess probe amounts may be used to ensure that all binding sites are occupied.

If more than one probe is used, they are preferably chosen so that they bind to different regions of the miRNA, and therefore cannot compete with each other for binding to the miRNA. The probes are also labeled with distinguishable detectable labels (i.e., the detectable label on the first probe is distinct from that on the second probe). Once the probes are allowed to bind to the miRNA (if it is present in the sample), the sample is analyzed for coincident emission signals. For example, a miRNA bound by both probes is manifest as overlapping emission signals from the bound probes. This detection is accomplished using a single molecule detection or analysis system. A single molecule detection or analysis system is a system capable of detecting and analyzing individual, preferably intact, molecules.

The method is particularly suited to detecting miRNA in a rare or small sample (e.g., a nanoliter volume sample) or in a sample where miRNA concentration is low. The invention allows more than one and preferably several different miRNA to be detected simultaneously, thereby conserving sample. In other words, the method is capable of a high degree of multiplexing. For example, the degree of multiplexing may be 2 (i.e., 2 miRNA can be detected in a single analysis), 3, 4, 5, 6, 7, 8, 9, 10, at least 20, at least 50, at least 100, at least 200, at least 300, at least 400, at least 500, or higher. Each miRNA is detected using a particular probe pair where preferably each member of the probe pair is specific to the miRNA (or at a minimum, one member of the pair is specific to the miRNA) and each probe used in an analysis is labeled with a distinguishable label. Thus, a plurality of miRNA may be detected and analyzed. As used herein, a plurality is an amount greater than two but less than infinity. A plurality is sometimes less than a million, less than a thousand, less than a hundred, or less than ten.

Probes

A probe is a molecule that specifically binds to a target of interest. The nature of the probe will depend upon the application and may also depend upon the nature of the target. Specific binding, as used herein, means the probe demonstrates greater affinity for its target than for other molecules (e.g., based on the sequence or structure of the target). The probe may bind to other molecules, but preferably such binding is at or near background levels. For example, it may have at least 2-fold, 5-fold, 10-fold or higher affinity for the desired target than for another molecule. Probes with the greatest differential affinity are preferred in most embodiments, although they may not be those with the greatest affinity for the target.

Probes can be virtually any compound that binds to a target with sufficient specificity. Examples include nucleic acids that bind to complementary nucleic acid targets via Watson-Crick and/or Hoogsteen binding (as discussed herein), aptamers that bind to nucleic acid targets due to structure rather than complementarity of sequence of the target, antibodies, etc. It is to be understood that although many of the exemplifications provided herein relate to nucleic acid probes, the invention is not so limited and other probes are envisioned.

“Sequence-specific” when used in the context of a probe means that the probe recognizes a particular linear arrangement of nucleotides or derivatives thereof. In preferred embodiments, the sequence-specific probe is itself composed of nucleic acid elements such as DNA, RNA, PNA and LNA elements or combinations thereof (as discussed herein). In preferred embodiments, the linear arrangement includes contiguous nucleotides or derivatives thereof that each binds to a corresponding complementary nucleotide in the probe. In some embodiments, however, the sequence may not be contiguous as there may be one, two, or more nucleotides that do not have corresponding complementary residues on the probe, and vice versa.

Any molecule that is capable of recognizing a nucleic acid with structural or sequence specificity can be used as a sequence-specific probe. In most instances, such probes will be nucleic acids themselves and will form at least a Watson-Crick bond with the target. In other instances, the nucleic acid probe can form a Hoogsteen bond with the nucleic acid target, thereby forming a triplex. A nucleic acid probe that binds by Hoogsteen binding enters the major groove of a nucleic acid target and hybridizes with the bases located there. In some embodiments, the nucleic acid probes can form both Watson-Crick and Hoogsteen bonds with the target. BisPNA probes, for instance, are capable of both Watson-Crick and Hoogsteen binding to a nucleic acid.

The length of the probe can also determine the specificity of binding. The energetic cost of a single mismatch between the probe and its target is relatively higher for shorter sequences than for longer ones. Therefore, hybridization of smaller nucleic acid probes is more specific than is hybridization of longer nucleic acid probes to the same target because the longer probes can embrace mismatches and still continue to bind to the target. One potential limitation to the use of shorter probes however is their inherently lower stability at a given temperature and salt concentration. One way of avoiding this latter limitation involves the use of bisPNA probes which bind shorter sequences with sufficient hybrid stability.

Notwithstanding these provisos, the nucleic acid probes of the invention can be any length ranging from at least 4 nucleotides to in excess of 1000 nucleotides. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 11 nucleotides, at least 12 nucleotides, at least 13 nucleotides, at least 14 nucleotides, at least 15 nucleotides, at least 20 nucleotides, or at least 25 nucleotides, or more, in length.

In some important embodiments, miRNA are detected using two or more probes. If two probes are used, each probe may be labeled at one of its ends such that when hybridized to the miRNA target, one probe is labeled at its 5′ end while the other is labeled at its 3′ end. The combined length of the probes may be longer than the total length of the miRNA. For example, if the miRNA target is 22 bases long, then each of the probes may be 12, 13 or more bases in length. Hybridization of such probes is intended to yield a duplex with a one, two or more base overhang at both ends. The bases to which the detectable labels are conjugated preferably are not themselves hybridized to complementary bases in the miRNA target. The use of longer probe pairs as described above has several advantages. First, it serves to stabilize the penultimate and final base pairings in the duplex, presumably due to an increased stability caused by nearest neighbor interactions. Second, the additional separation from the labels will reduce quenching and/or FRET between the labels. Third, the increase in size of the duplex will aid the size-based separation of the duplex from the unreacted targets and probes. In some embodiments, the overhangs may comprise an adenosine, thymine, guanine or cytosine, although modified bases such as LNA, iso-C or iso-G may also be used.

It should be understood that not all residues of the probe need to hybridize to complementary residues in the nucleic acid target, although this is preferred. For example, the probe may be 50 residues in length, yet only 45 of those residues hybridize to the nucleic acid target. Preferably, the residues that hybridize are contiguous with each other.

The length of the probe may also be represented as a proportion of the length of the miRNA to which it binds specifically. For example, the probe length may be at least 10%, at least 20%, at least 30%, at least 40%, or at least 50% the length of its target miRNA, or longer.

The probes are preferably single-stranded, but they are not so limited. For example, when the probe is a bisPNA it can adopt a secondary structure with the nucleic acid target (e.g., the miRNA) resulting in a triple helix conformation, with one region of the bisPNA clamp forming Hoogsteen bonds with the backbone of the tailed miRNA and another region of the bisPNA clamp forming Watson-Crick bonds with the nucleotide bases of the tailed miRNA.

The nucleic acid probe hybridizes to a complementary sequence within the miRNA. The specificity of binding can be manipulated based on the hybridization conditions. For example, salt concentration and temperature can be modulated in order to vary the range of sequences recognized by the nucleic acid probes. Those of ordinary skill in the art will be able to determine optimum conditions for a desired specificity.

Nucleic Acids and Derivatives Thereof

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The organic bases include adenine, uracil, guanine, thymine, cytosine and inosine. The nucleic acids may be single- or double-stranded. Nucleic acids can be obtained from natural sources, or can be synthesized using a nucleic acid synthesizer.

As used herein with respect to linked units of a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting for example the individual units of a particular nucleic acid, are most common. Natural linkages include, for instance, amide, ester and thioester linkages. The individual units of a nucleic acid may be linked, however, by synthetic or modified linkages. Nucleic acids where the units are linked by covalent bonds will be most common but those that include hydrogen bonded units are also embraced by the invention. It is to be understood that all possibilities regarding nucleic acids apply equally to nucleic acid tails, nucleic acid probes and capture nucleic acids.

In some embodiments, the invention embraces nucleic acid derivatives as nucleic acid probes and the like. As used herein, a “nucleic acid derivative” is a non-naturally occurring nucleic acid or a unit thereof. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

Nucleic acid probes can be stabilized in part by the use of backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, nucleic acid probes may include a peptide nucleic acid (PNA), a bisPNA clamp, a pseudocomplementary PNA, a locked nucleic acid (LNA), DNA, RNA, or co-nucleic acids of the above such as DNA-LNA co-nucleic acids (as described in co-pending U.S. patent application having Ser. No. 10/421,644 and publication number US 2003-0215864 A1 and published Nov. 20, 2003, and PCT application having serial number PCT/US03/12480 and publication number WO 03/091455 A1 and published Nov. 6, 2003, filed on Apr. 23, 2003), or co-polymers thereof (e.g., a DNA-LNA co-polymer).

In some important embodiments, the nucleic acid probe is a LNA/DNA chimeric probe. LNA content may vary from more than 0% to less than 100%, and may include at least 5%, at least 10%, at least 20%, at least 30%, at least 40%, at least 50%, at least 60%, at least 70%, at least 80%, at least 90%, at least 95%, or at least 99%. In some embodiments, 10- or 11-mer probes may contain on average about 3-4 LNAs, for example.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA- or RNA-based probes.

PNAs are synthesized from monomers connected by a peptide bond (Nielsen, P. E. et al. Peptide Nucleic Acids, Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

PNA has a charge-neutral backbone, and this attribute leads to fast hybridization rates of PNA to DNA (Nielsen, P. E. et al. Peptide Nucleic Acids, Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bisPNA and pseudocomplementary PNA (pcPNA).

The structure of PNA/DNA complex depends on the particular PNA and its sequence. Single stranded PNA (ssPNA) binds to single-stranded DNA (ssDNA) preferably in anti-parallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids, Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bisPNA/DNA complex is still stable as it forms a hybrid with twice as many (e.g., a 16 bp) base pairings overall. The bisPNA structure further increases specificity of their binding. As an example, binding to an 8 bp site with a probe having a single base mismatch results in a total of 14 bp rather than 16 bp.

Pseudocomplementary PNA (pcPNA) (Izvolsky, K. I. et al., Biochemistry 10908-10913 (2000)) involves two single-stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diamiopurine (D) instead of adenine and 2-thiouracil (^{S}U) instead of thymine. While D and ^{S}U are still capable of hybridization with T and A respectively, their self-hybridization is sterically prohibited.

Locked nucleic acids (LNA) are modified RNA nucleotides. (See, for example, Braasch and Corey, Chem. Biol., 2001, 8(1):1-7.) LNAs form hybrids with DNA which are at least as stable as PNA/DNA hybrids. Therefore, LNA can be used just as PNA molecules would be. LNA binding efficiency can be increased in some embodiments by adding positive charges to it.

Commercial nucleic acid synthesizers and standard phosphoramidite chemistry are used to make LNAs. Therefore, production of mixed LNA/DNA sequences is as simple as that of mixed PNA/peptide sequences. Naturally, most of biochemical approaches for nucleic acid conjugations are applicable to LNA/DNA constructs.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine (particularly useful if positive charges are desired in the PNA), and the like. Various PNA modifications are known and probes incorporating such modifications are commercially available from sources such as Boston Probes, Inc.

Labeling of Sequence-Specific Probes

The probes are detectably labeled (i.e., they comprise a detectable label). A detectable label is a moiety, the presence of which can be ascertained directly or indirectly. Generally, detection of the label involves the creation of a detectable signal such as for example an emission of energy. The label may be of a chemical, lipid, peptide or nucleic acid nature although it is not so limited. The nature of label used will depend on a variety of factors, including the nature of the analysis being conducted, the type of the energy source and detector used. The label should be sterically and chemically compatible with the constituents to which it is bound.

The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.).

There are several known methods of direct chemical labeling of DNA. (Hermanson, G. T., Bioconjugate Techniques, Academic Press, Inc., San Diego, 1996; Roget et al., 1989; Proudnikov and Mirabekov, Nucleic Acid Research, 24:4535-4532, 1996.) One of the methods is based on the introduction of aldehyde groups by partial depurination of DNA. Fluorescent labels with an attached hydrazine group are efficiently coupled with the aldehyde groups and the hydrazine bonds are stabilized by reduction with sodium labeling efficiencies around 60%. The reaction of cytosine with bisulfite in the presence of an excess of an amine fluorophore leads to transamination at the N4 position (Hermanson, 1996). Reaction conditions such as pH, amine fluorophore concentration, and incubation time and temperature affect the yield of products formed. At high concentrations of the amine fluorophore (3M), transamination can approach 100% (Draper and Gold, 1980).

It is also possible to synthesize nucleic acids de novo (e.g., using automated nucleic acid synthesizers) using fluorescently labeled nucleotides. Such nucleotides are commercially available from suppliers such as Amersham Pharmacia Biotech, Molecular Probes, and New England Nuclear/Perkin Elmer.

Generally the detectable label can be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo)benzoic acid (DABCYL), 5-(2′-aminoethyl)aminonaphthalene-1-sulfonic acid (EDANS), 4-acetamido-4′-isothiocyanatostilbene-2,2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′, 6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl)aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., P³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle (such as quantum dots described for example in U.S. Pat. No. 6,207,392 and commercially available from Quantum Dot Corporation and Evident Technologies), a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like. Antibody fragments include Fab, F(ab)₂, Fd and antibody fragments which include a CDR3 region.

In some embodiments, the first and second probes may be labeled with fluorophores that form a fluorescence resonance energy transfer (FRET) pair. In this case, one excitation wavelength is used to excite fluorescence of donor fluorophores. A portion of the energy absorbed by the donors can be transferred to acceptor fluorophores if they are close enough spatially to the donor molecules (i.e., the distance between them must approximate or be less than the Forster radius or the energy transfer radius). Once the acceptor fluorophore absorbs the energy, it in turn fluoresces in its characteristic emission wavelength. Since energy transfer is possible only when the acceptor and donor are located in close proximity, acceptor fluorescence is unlikely if both probes are not bound to the same miRNA. Acceptor fluorescence therefore can be used to determine presence of miRNA.

It is to be understood however that if a FRET fluorophore pair is used, coincident binding of the pair to a single target is detected by the presence or absence of a signal rather than a coincident detection of two signals.

A FRET fluorophore pair is two fluorophores that are capable of undergoing FRET to produce or eliminate a detectable signal when positioned in proximity to one another. Examples of donors include Alexa 488, Alexa 546, BODIPY 493, Oyster 556, Fluor (FAM), Cy3 and TMR (Tamra). Examples of acceptors include Cy5, Alexa 594, Alexa 647 and Oyster 656. Cy5 can work as a donor with Cy3, TMR or Alexa 546, as an example. FRET should be possible with any fluorophore pair having fluorescence maxima spaced at 50-100 nm from each other. The FRET embodiment can be coupled with another label on the target miRNA such as a backbone label, as discussed below.

The miRNA target may be additionally labeled with a backbone label. These labels generally label nucleic acids in a sequence non-specific manner. In these embodiments, the miRNA may be detected by the coincident signals from the backbone label and one or more of the bound probes. Examples of backbone labels (or stains) include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

Therefore, some embodiments of the invention embrace three color coincidence. In these embodiments, single or multiple lasers may be used. For example, three different lasers may be used for excitation at the following wavelengths: 488 nm (blue), 532 nm (green), and 633 nm (red). These lasers excite fluorescence of Alexa 488, TMR (tetramethylrhodamine), and TOTO-3 fluorophores, respectively. Fluorescence from all these fluorophores can be detected independently. As an example of fluorescence strategy, one sequence-specific probe may be labeled with Alexa 488 fluorophore, a second sequence-specific probe may be labeled with TMR, and the miRNA backbone may be labeled with TOTO-3. TOTO-3 is an intercalating dye that non-specifically stains nucleic acids in a length-proportional manner. Another suitable set of fluorophores that can be used is the combination of POPO-1, TMR and Alexa 647 (or Cy5) which are excited by 442, 532 and 633 nm lasers respectively.

Conjugation, Linkers and Spacers

As used herein, “conjugated” means two entities stably bound to one another by any physicochemical means. It is important that the nature of the attachment is such that it does not substantially impair the effectiveness of either entity. Keeping these parameters in mind, any covalent or non-covalent linkage known to those of ordinary skill in the art is contemplated unless explicitly stated otherwise herein. Non-covalent conjugation includes hydrophobic interactions, ionic interactions, high affinity interactions such as biotin-avidin and biotin-streptavidin complexation and other affinity interactions. Such means and methods of attachment are known to those of ordinary skill in the art. Conjugation can be performed using standard techniques common to those of ordinary skill in the art.

The various components described herein can be conjugated by any mechanism known in the art. For instance, functional groups which are reactive with various labels include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

Linkers and/or spacers may be used in some instances. Linkers can be any of a variety of molecules, preferably nonactive, such as nucleotides or multiple nucleotides, straight or even branched saturated or unsaturated carbon chains of C₁-C₃₀, phospholipids, amino acids, and in particular glycine, and the like, whether naturally occurring or synthetic. Additional linkers include alkyl and alkenyl carbonates, carbamates, and carbamides. These are all related and may add polar functionality to the linkers such as the C₁-C₃₀ previously mentioned. As used herein, the terms linker and spacer are used interchangeably.

A wide variety of spacers can be used, many of which are commercially available, for example, from sources such as Boston Probes, Inc. (now Applied Biosystems). Spacers are not limited to organic spacers, and rather can be inorganic also (e.g., —O—Si—O—, or O—P—O—).

Additionally, they can be heterogeneous in nature (e.g., composed of organic and inorganic elements). Essentially, any molecule having the appropriate size restrictions and capable of being linked to the various components such as fluorophore and probe can be used as a linker. Examples include the E linker (which also functions as a solubility enhancer), the X linker which is similar to the E linker, the 0 linker which is a glycol linker, and the P linker which includes a primary aromatic amino group (all supplied by Boston Probes, Inc., now Applied Biosystems). Other suitable linkers are acetyl linkers, 4-aminobenzoic acid containing linkers, Fmoc linkers, 4-aminobenzoic acid linkers, 8-amino-3,6-dioxactanoic acid linkers, succinimidyl maleimidyl methyl cyclohexane carboxylate linkers, succinyl linkers, and the like. Another example of a suitable linker is that described by Haralambidis et al. in U.S. Pat. No. 5,525,465, issued on Jun. 11, 1996. The length of the spacer can vary depending upon the application and the nature of the components being conjugated

The linker molecules may be homo-bifunctional or hetero-bifunctional cross-linkers, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl)suberate, bis[2-(succinimidooxycarbonyloxy)ethyl]sulfone, disuccinimidyl suberate, disuccinimidyl tartarate, dimethyl adipimate.2 HCl, dimethyl pimelimidate.2 HCl, dimethyl suberimidate.2 HCl, and ethylene glycolbis-[succinimidyl-[succinate]]. Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide.2 HCl, 4-(4-N-maleimidophenyl)-butyric acid hydrazide.2 HCl, and 3-[2-pyridyldithio]propionyl hydrazide. The cross-linkers are bis-[β-4-azidosalicylamido)ethyl]disulfide and glutaraldehyde.

Amine or thiol groups may be added at any nucleotide of a synthetic nucleic acid so as to provide a point of attachment for a bifunctional cross-linker molecule. The nucleic acid may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

In some instances, it may be desirable to use a linker or spacer comprising a bond that is cleavable under certain conditions. For example, the bond can be one that cleaves under normal physiological conditions or that can be caused to cleave specifically upon application of a stimulus such as light, whereby the conjugated entity is released leaving its conjugation partner intact. Readily cleavable bonds include readily hydrolyzable bonds, for example, ester bonds, amide bonds and Schiff's base-type bonds. Bonds which are cleavable by light are known in the art.

Detection Systems

Nucleic acids may be analyzed using a single molecule analysis system. A single molecule analysis system is capable of analyzing single, preferably intact, molecules separately from other molecules. Such a system is sufficiently sensitive to detect signals emitting from a single molecule and its bound probes. The system may be a linear molecule analysis system in which single molecules are analyzed in a linear manner (i.e., starting at a point along the polymer length and then moving progressively in one direction or another). Many of the methods provided herein do not require linear analysis of miRNA.

The system is preferably not an electrophoretic method and thus is sometimes referred to as a non-electrophoretic single molecule detection (or analysis) system. Such systems do not rely on gel electrophoresis or capillary electrophoresis to separate molecules from each other.

An example of a single molecule detection/analysis system is the Trilogy™ instrument which is based on the Gene Engine™ technology described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The Gene Engine™ system allows single polymers to be passed through an interaction station, whereby the units of the polymer or labels of the compound are interrogated individually in order to determine whether there is a detectable label conjugated to the target. Interrogation involves exposing the label to an energy source such as optical radiation of a set wavelength. In response to the energy source exposure, the detectable label emits a detectable signal. The mechanism for signal emission and detection will depend on the type of label sought to be detected.

The Trilogy™ system is a single molecule confocal fluorescence detection platform. The platform enables four-color fluorescent detection in a microfluidic flow stream with engineering modifications to automate sample handling and delivery. In this embodiment, photons emitted by the fluorescently tagged molecules pass through the dichroic mirror and are band-pass filtered to remove stray laser light and any Rayleigh or Raman scattered light. The emission is focused and filtered through 100 micrometer pinholes of multi-mode fiber optic cables coupled to single photon counting modules. A high-speed data acquisition card is used to store photon counts from each channel using a 10 kHz sampling rate. It should be noted that this system has single fluorophore detection sensitivity of four spectrally distinct fluorophores. The Trilogy™ provides real-time counting of individually labeled molecules in a nanoliter interrogation zone. The system detects labeled molecules at low femtomolar concentrations and displays a dynamic range over 4+ logs. The system can accommodate standard sample carriers such as but not limited to 96 well plates or microcentrifuge (e.g., Eppendorf) tubes. The sample volumes may be on the order of microliters (e.g., 1 ul volume).

The systems described herein will encompass at least one detection system. The nature of such detection systems will depend upon the nature of the detectable label. The detection system can be selected from any number of detection systems known in the art. These include an electron spin resonance (ESR) detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

The entire contents of all of the references (including literature references, issued patents, published patent applications, and co-pending patent applications) cited throughout this application are expressly incorporated by reference herein.

What is claimed is:

1. A method for detecting a condition comprising
determining a level of a miRNA in a test tissue sample using a first and a second miRNA specific probes that are differentially labeled, and
comparing the level of the miRNA in the test tissue sample to a level of the miRNA in a control tissue sample,
wherein a difference in the level of the miRNA in the test and the control tissue samples is indicative of the condition, and
wherein the level of the miRNA is determined by coincidence detection of differentially labeled probes.

2. The method of claim 1, wherein the difference in the level of the miRNA in the test and the control tissue samples is a greater level of miRNA in the test tissue sample.

3. The method of claim 1, wherein the difference in the level of the miRNA in the test and the control tissue samples is a greater level of miRNA in the control tissue sample.

4. The method of claim 1, wherein coincidence detection comprises detecting binding of a first miRNA-specific probe labeled with a first detectable label and a second miRNA-specific probe labeled with a second detectable label distinguishable from the first detectable label to the miRNA.

5. The method of claim 1, wherein coincidence detection comprises subtracting a random coincidence estimate from a raw coincidence count.

6. The method of claim 1, wherein the test tissue sample is a breast tissue sample, a cervical tissue sample, an ovarian tissue sample, or a prostate tissue sample.

7. The method of claim 1, wherein the condition is cancer.

8. The method of claim 7, wherein the cancer is breast cancer, cervical cancer, colon cancer, ovarian cancer, or prostate cancer.

9. The method of claim 1, wherein the condition is cirrhosis, and the test issue sample is a liver tissue sample.

10. The method of claim 1, wherein the miRNA is mir-143 or mir-145.

11. The method of claim 1, wherein coincidence detection comprises use of a quencher probe.

12. The method of claim 1, wherein the miRNA is present at a concentration of 1-100 femtomolar.

13. The method of claim 1, wherein the miRNA is present at a concentration of 1-10 femtomolar.
